(19) United States
(12) Patent Application Publication
(10) Pub. No.: US 2023/0348599 A1
(43) Pub. Date: Nov. 2, 2023

(54) METHODS FOR TREATING GLIOBLASTOMA

(71) Applicant: BOARD OF REGENTS, THE UNIVERSITY OF TEXAS SYSTEM, Austin, TX (US)

(72) Inventors: Padmanee SHARMA, Houston, TX (US); James ALLISON, Houston, TX (US); Sreyashi BASU, Houston, TX (US)

(73) Assignee: BOARD OF REGENTS, THE UNIVERSITY OF TEXAS SYSTEM, Austin, TX (US)

(21) Appl. No.: 17/786,917

(22) PCT Filed: Dec. 17, 2020
(86) PCT No.: PCT/US2020/065724

§ 371 Date: Jun. 17, 2022

Related U.S. Application Data
(60) Provisional application No. 62/950,509, filed on Dec. 19, 2019.

(51) Int. Cl. C07K 16/28; A61K 45/06; G01N 33/574; A61P 35/00

(52) U.S. Cl. CPC C07K 16/2818; C07K 16/2896; A61K 45/06; G01N 33/57484; A61P 35/00; G01N 2333/70596; G01N 2800/52

(57) ABSTRACT

The current disclosure provides for novel therapeutic methods by identifying glioblastoma patient populations that may be treated effectively by immunotherapies. Also provided are therapies that may be used in combination of immune checkpoint therapy (ICB) to increase the effectiveness of the therapy. Aspects of the disclosure relate to a method of treating glioblastoma in a subject comprising administering to the subject immune checkpoint blockade (ICB) therapy after the subject has been determined to have low expression of CD73 in a biological sample from the subject.

This application claims priority to U.S. Provisional Application No. 62/950,509, filed Dec. 19, 2019, which is hereby incorporated by reference in its entirety

### BACKGROUND OF THE INVENTION

### II. Field of the Invention

This invention relates to the field of biotechnology and therapeutic treatment methods.

### III. Background

Tremendous advances were made in cancer therapy in the past decade through the use of targeted therapy and immune therapy. By blocking immune inhibitory ligand-receptor interactions involving CTLA-4 and PD-1, checkpoint blockade immunotherapy relieves T lymphocytes of major inhibitory signals, thus potentiating underlying T cell-mediated anti-tumor immune activity. However, ubiquitous relief of inhibitory signals systemically can also activate T lymphocytes reactive against self-antigens, leading to loss of self-tolerance and immune-related adverse events. Patients who develop high-grade toxicities commonly require either temporary or permanent discontinuation of treatment, and may require prolonged periods of heavy immunosuppression in order to manage their toxicities. The high frequency of developing severe to life threatening toxicity to anti-CTLA-4 and/or anti-PD-1 therapy and the unpredictability with respect to whether a patient will respond has become a limiting factor for clinicians to prescribe this form of therapy.

While some factors associated with patient response to immune checkpoint inhibitor therapy have been discovered, there is a need in the art for predictors of toxicity due to immune checkpoint blockade therapy and predictors of responders to immune checkpoint blockade therapy. Stratifying patients into those that are likely and unlikely to respond to checkpoint blockade therapy, based on one or more biomarkers, will provide for more effective and therapeutic treatment methods for patients, since patients can be provided with the most effective therapy before further spreading of the disease.

### SUMMARY OF THE INVENTION

The current disclosure provides for novel therapeutic methods by identifying glioblastoma patient populations that may be treated effectively by immunotherapies. Also provided are therapies that may be used in combination of immune checkpoint therapy (ICB) to increase the effectiveness of the therapy. Accordingly, aspects of the disclosure relate to a method of treating glioblastoma in a subject comprising administering to the subject immune checkpoint blockade (ICB) therapy after the subject has been determined to have low expression of CD73 in a biological sample from the subject. Further aspects relate to a method of treating glioblastoma in a subject comprising administering to the subject an agent selected from a CD73 inhibitor, a CD39 inhibitor, or an A2AR antagonist after the subject has been determined to have high expression of CD73 in a biological sample from the subject.

Further aspects relate to a method for predicting a response to ICB therapy in a subject having glioblastoma, the method comprising: (a) determining the expression level of CD73 in a sample from the subject; (b) comparing the expression level of CD73 in a sample from the subject to a control; and (c) predicting that the subject will respond to the ICB therapy after (i) a decreased expression level of CD73 is detected in a biological sample from the subject as compared to a control, wherein the control represents an expression level of CD73 in a biological sample from a subject that has been determined to not respond to ICB therapy; or (ii) a decreased or a non-significantly different expression level of CD73 is detected in a biological sample from the subject as compared to a control, wherein the control represents an expression level of CD73 in a biological sample from a subject that has been determined to respond to ICB therapy; or (d) predicting that the subject will not respond to the ICB therapy after (i) an increased expression level of CD73 is detected in a biological sample from the subject as compared to a control, wherein the control represents an expression level of CD73 in a biological sample from a subject that has been determined to respond to ICB therapy; or (ii) an increase or a non-significantly different expression level of CD73 is detected in a biological sample from the subject as compared to a control, wherein the control represents an expression level of CD73 in a biological sample from a subject that has been determined to not respond to ICB therapy.

Yet further aspects relate to a method comprising detecting CD73 in a biological sample from a subject with glioblastoma. In some embodiments, a low level of CD73 expression is detected. In some embodiments, a high level of CD73 expression is detected.

In some embodiments, the biological sample comprises isolated immune cells. In some embodiments, the biological sample comprises isolated macrophages. In some embodiments, the biological samples comprises a serum sample. In some embodiments, the biological sample comprises an isolated fraction of immune cells. In some embodiments, the biological sample comprises a biopsy. In some embodiments, the biological samples comprises a sample comprising tissue cells and immune cells. In some embodiments, the tissue comprises cells from a glioblastoma tumor. In some embodiments, the expression of CD73 is determined to be low in immune cells, as compared to a control. In some embodiments, the expression of CD73 is determined to be high in immune cells, as compared to a control. In some embodiments, the high expression level of CD73 or the low expression level of CD73 was determined in the biological sample from the subject by comparing the expression level of CD73 in the biological sample from the subject to a control. In some embodiments, the low expression refers to low number of CD73+ immune cells detected in the biological sample from the subject, as compared to a control. In some embodiments, the high expression refers to high number of CD73+ immune cells detected in the biological sample from the subject, as compared to a control. For example, low expression may refer to a low number of CD73+ immune cells detected in a biological sample, such as a biopsy, as compared to a standard, baseline, or control, wherein said standard, baseline, or control represents the number of CD73+ immune cells detected in a biological sample from a subject that has been determined to be responsive to immune therapy, or is within 0.5, 1, 2, or 3 standard deviations, or is not significantly different to the control. Similarly, high expression may refer to a high number of CD73+ immune cells detected in a biological sample, such as a biopsy, as compared to a standard, baseline, or control, wherein said standard, baseline, or control represents the number of CD73+ immune cells detected in a biological sample from a subject that has been determined to be responsive to immune therapy, or is at least 1.5, 2, 3, 4, 5, 6, 10, 20, 100, 500, or 1000× more than the control. In some embodiments, the biological sample from the subject may be fractionated to isolate immune cells from other cells. In some embodiments, the biological sample is fractionated to isolate immune cells from tumor cells, and the expression level of CD73 or amount of CD73+ cells is determined in the isolated fraction. In some embodiments, the biological sample does not comprise tumor cells, is essentially free of tumor cells, or is a fraction in which immune cells have been enriched from and tumor cells have been depleted.

In some embodiments, the ICB therapy comprises a monotherapy or a combination ICB therapy. In some embodiments, the subject has been determined to be a candidate for ICB therapy. In some embodiments, the subject is currently being treated with ICB therapy, has received at least one ICB therapy. In some embodiments, the subject has not been treated with ICB therapy. In some embodiments, the subject has been determined to be non-responsive to the previous treatment.

In some embodiments of the disclosure, the method comprises or further comprises treating a subject with ICB therapy. In some embodiments, the subject is one that is predicted to respond to the ICB therapy based on the detected level of CD73 in a biological sample from the subject. In some embodiments, the ICB therapy comprises an inhibitor of PD-1, PDL1, PDL2, CTLA-4, B7-1, and/or B7-2. In some embodiments, the ICB therapy comprises an anti-PD-1 monoclonal antibody and/or an anti-CTLA-4 monoclonal antibody. In some embodiments, the ICB therapy comprises one or more of nivolumab, pembrolizumab, pidilizumab, ipilimumab or tremelimumab.

In some embodiments, the method further comprises administering at least one additional anticancer treatment. In some embodiments, at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy. In some embodiments, the method further comprises administration of ICB therapy to the subject.

In some embodiments, the control comprises a cut-off value or a normalized value. In some embodiments, the expression level comprises a normalized level of expression. In some embodiments, CD73 expression was detected by an immunoassay. In some embodiments, the low expression level comprises a normalized level of expression that is determined to be decreased compared to a control. In some embodiments, the low expression level comprises a normalized level of expression that is determined to be increased compared to a control.

In some embodiments, the CD73 inhibitor, CD39 inhibitor, or A2AR antagonist is administered prior to the ICB therapy. In some embodiments, the ICB therapy and CD73 inhibitor, CD39 inhibitor, or A2AR antagonist are administered simultaneously. In some embodiments, the CD73 inhibitor, CD39 inhibitor, or A2AR antagonist is administered at least 1, 2, 3, 4, 5, 6, 7, 8, 9, 10, 11, 12, 13, 14, 15, 16, 17, 18, 19, 20, 21, 22, 23, or 24 hours, days, or weeks (or any range derivable therein) prior to the ICB therapy. In some embodiments, the CD73 inhibitor, CD39 inhibitor, or A2AR antagonist is administered within at least 1, 2, 3, 4, 5, 6, 7, 8, 9, 10, 11, 12, 13, 14, 15, 16, 17, 18, 19, 20, 21, 22, 23, or 24 hours, days, or weeks (or any range derivable therein) of administration of the ICB therapy.

In some embodiments, the CD73 or CD39 inhibitor comprises an anti-CD73 or an anti-CD39 antibody, respectively. In some embodiments, the antibody comprises a blocking antibody and/or induces antibody-dependent cellular cytotoxicity. In some embodiments, the A2AR antagonist comprises ATL-444, Istradefylline (KW-6002), MSX-3, Preladenant (SCH-420,814), SCH-58261, SCH-412,348, SCH-442,416, ST-1535, Caffeine, VER-6623, VER-6947, VER-7835, Vipadenant (BIIB-014), ZM-241,385, or combinations thereof.

In some embodiments, the method further comprises comparing the expression level of CD73 detected to a control. In some embodiments, the control comprises a biological sample from a subject that does not respond to ICB therapy. In some embodiments, the control comprises a biological sample from a subject that responds to ICB therapy. In some embodiments, the subject is determined to have a higher expression level than the control. In some embodiments, the subject is determined to have a lower expression level than the control. In some embodiments, the subject is determined to have a level of expression that is not significantly different than the control.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-F. Identification of Tumor infiltrating leukocyte phenotypes. TILs were analyzed by CyTOF and identified using the PhenoGraph algorithm on viable CD45⁺ cells. A, Box-plots indicating frequency of CD3, CD4, CD8 or CD68 positive cells and CD4⁺FoxP3⁺ cells from live singlets obtained by manual gating of mass cytometry data (n=66). In all the box plots depicted, boxes indicate interquartile range with central bar indicating median and whiskers indicating the range. Individual patients are represented with dots. p values were computed by Mann-Whitney tests (two sided). Q values were calculated using the p.adjust function. q<0.05 was considered statistically significant. B, Heatmap depicting normalized expression of different immune markers by our PhenoGraph-based clustering approach on CD45+ cells obtained from NSCLC (n=11), RCC (n=11), CRC (n=11), PCa (n=5) and GBM (n=7) patients. The color bar on the right indicates the leukocyte lineage of the respective meta-cluster (Myeloid: CD3⁻CD68⁺; T cell: CD3⁺; NK cell: CD3⁻CD56+). Bar graphs on the right indicate the relative frequency of the respective meta-clusters. C, Box-plots indicating Shannon entropy of the distribution of tumor types in immune meta-clusters. Shannon entropy was computed for an empirical distribution of tumor across 1000 cells. This procedure was repeated 1000 times per cluster in order to bootstrap cluster size-corrected standard errors of entropy (n=1000). Boxplots of entropy values in each cluster, ordered by mean entropy. Dashed line indicating the expected entropy value, if in-cluster tumor type distribution matches tumor type distribution of all cells in the dataset. D, Box-plots indicating frequencies of the respective CD4 and CD8 T cell meta-clusters across tumor types. (Number of patients: GBM=7, NSCLC=11, RCC=11, CRC=11 PCa=5). Kruskal-Wallis tests were performed for the 14 metaclusters and corrected for multiple comparisons using the Benjamini and Hochberg (BH) method. E, Stacked bar graph visualizing meta-clusters frequencies in individual patients using the color-code indicated on the right. Dendrogram on the left indicating hierarchical clustering of patient meta-cluster frequencies. Black frames highlighting patient subgroups identified by this clustering approach. Color bar on the left indicating tumor types of the individual patients using the color code indicated below. F, Box-plots indicating T cell meta-cluster frequencies across the patient subgroups identified in E. Group I=11, group II=8, group III=9. In all the box plots depicted, boxes indicate interquartile range with central bar indicating median and whiskers indicating the range. Individual patients are represented with dots. Kruskal-Wallis tests were used to compare across the subgroups. Mann-Whitney tests were used for pairwise comparisons. Significant pairwise comparisons are indicated (FDR=5%).

FIG. 2A-E. FIG. 2. CD73^{hi} macrophages are specifically present in GBM. Myeloid cells (CD3 CD68⁺) were analyzed by CyTOF in patients of multiple tumor types and further characterized by sc-RNA seq in GBM. A, Box-plots indicating frequencies of L1, L5, L8 and L17 meta-cluster across tumor types (number of patients: NSCLC=11, RCC=11, CRC=11, PCa=5, GBM=7). Q-values were calculated using Kruskal-Wallis tests (across different tumor types) and the Benjamini & Hochberg method. Pairwise comparisons were performed using Mann-Whitney U tests within and corrected for multiple comparisons using the Benjamini & Hochberg. Significant pairwise comparisons are indicated (FDR=5%). B, TILs from untreated GBM tumors (n=4) were analyzed by sc-RNA seq and identified using the MAGIC algorithm. Heatmap indicating normalized expression of selected markers in leukocyte clusters identified by MAGIC. Black arrows indicate the CD73^{hi} myeloid cell clusters. C, Upper top panel: t-SNE maps depicting cluster phenotypes and relative expression levels of CD73 on a single cell level with color legend on the right. Oval area highlighting CD73^{hi} macrophage clusters (R3, R7, R14 and R17). Lower bottom panel: t-SNE maps indicating relative expression levels of a blood-derived macrophage gene signature and microglial gene signature at a single cell level with color legend on the right (n=4). D, Heatmap indicating normalized expression of chemokine receptors on CD73^{hi} macrophage clusters identified by MAGIC. Black arrows indicate the CD73^{hi} myeloid cell clusters. E, Upper panel: t-SNE maps indicating relative expression levels of immunosuppressive and immunostimulatory gene signature at a single cell level. Lower bottom panel: t-SNE maps indicating relative expression levels of a hypoxia-induced gene signature, (n=4).

FIG. 3A-G. FIG. 3. CD73^{hi} myeloid cells persist after anti-PD-1 therapy and correlates with reduced overall survival in TCGA-GBM cohort. CD73^{hi} macrophage gene signature of differentially expressed genes (z>3.0, 45 genes) (Supplementary Table 3). Heatmap indicating normalized expression of top differentially expressed genes in CD73^{hi} macrophages (z score >2.0) identified by MAGIC. B, Kaplan-Meier plot showing overall survival of GBM patients from the TCGA database with above (blue=high expression, number of patients: n=263) or below (red=low expression, number of patients: n=262) median expression of 45 genes signature derived in A. Log rank p value (two-sided) and hazard ratio (HR) displayed. Leukocyte phenotypes in single cell suspensions of tumors from immune-checkpoint naïve patients (untreated) and Pembrolizumab treated patients (pembro) were analyzed by mass cytometry and identified using the PhenoGraph algorithm on viable CD45⁺ cells. C, t-SNE map depicting degree of phenotypic similarity of GBM infiltrating leukocytes in pembrolizumab-treated (n=5) or untreated patients (n=7) at a single cell level. D, TILs from GBM tumors after treatment with pembrolizumab (n=5) or ICT naïve GBM patients (n=7) were analyzed by mass cytometry and identified using the PhenoGraph algorithm on viable CD45⁺ cells. Heatmap indicating normalized expression of selected markers on CD45⁺ meta-clusters identified by PhenoGraph. E-F, Stacked-bars indicating frequencies of CD73^{hi} myeloid meta-clusters and T cell clusters in pembrolizumab treated and untreated GBM patients. G, Representative heat map of transcriptome profiling using GSEA of tumor specimens from untreated (n=6) and anti-PD-1 treated (n=4) patients using customized 739-gene Nanostring panel.

FIG. 4A-D. Absence of CD73 enhances efficacy of ICT in murine model of GBM. A, Representative MRI images on day 14 of inoculation of GL-261 tumor line othotopically into CD73^{-/-} and wild-type mice with and without ICT treatment. Figures are representative of three independent experiments. B, Kaplan-Meier plot showing overall survival of wild-type and CD73^{-/-} (n˜10 mice) treated with anti-PD-1 alone, anti-PD-1 and anti-CTLA-4 or untreated mice orthotopically injected with GL-261 gliomas, p values were calculated using a logrank test (two sided). Please refer to Supplementary Table 2 for more details. C, Heatmap indicating intra-tumoral CD45+ immune populations as determined by FlowSOM in both WT and CD73^{-/-} mice bearing GBM tumors. Color code on the upper right indicates z-scored expression values. Legend on the lower right indicates cell types for each colored cluster. D, Box-plots indicating abundance ratio of leukocyte subsets (n=5 mice per group). Data representative of 2 independent experiments. Data in the box plots are means±SEM. P-values were calculated using Mann-Whitney U tests (two sided) for pairwise comparisons.

FIG. 5. Gating strategy for identification of immune cell subsets by manual gating. Contour plots indicating the gating strategy used to define manually gated CD3, CD4, CD8 and FoxP3 positive populations in FIG. 1a.

FIG. 6A-D. Heterogeneity of Tumor Infiltrating Leukocytes. A, Scatter plot indicating the absolute number of CD45+ live singlets of mass cytometry samples used for the multi-tumor comparison. Dashed line depicting the 600-cell threshold for sample inclusion. B, Stacked bars (left) depicting the distribution of the identified meta-cluster frequencies within different tumor types in the color code indicated below. t-SNE map of 10,000 randomly selected cells per tumor type colored by tumor type with color legend indicated on the right (right, top panel), or by meta-cluster (right, bottom panel) with color legend indicated in the left panel. C, Boxplots indicating CD45+ immune meta-cluster frequencies across tumor types from the PhenoGraph-based clustering approach in FIG. 1. (Number of patients, GBM=7, NSCLC=11, RCC=11, CRC=11, and PCa=5). In all the box plots depicted, boxes indicate interquartile range with central bar indicating median and whiskers indicating the range. Individual patients are represented with dots. D, Histograms depicting expression of immune markers on the respective meta-clusters indicated on the left related to FIG. 1d.

FIG. 7A-C. PD-1^{hi} T cells expand during immune checkpoint therapy in clinical responders. T cell phenotypes in PBMC suspensions from renal cell carcinoma (RCC) patients undergoing combined ipilimumab and nivolumab ICT were analyzed by mass cytometry and identified using the PhenoGraph algorithm on viable CD45+ cells (n=14). A, Heatmap indicating normalized expression of selected markers on CD45+ meta-clusters identified by PhenoGraph. B, CD4+ T cell cluster P33 and CD8+ T cell cluster P24 frequencies at pretreatment (T0) and after two cycles (T2) or four cycles (T4) of combination ICT in responders (n=7) and non-responders (n=7). P values were calculated using Mann-Whitney U tests (two-sided). Q values were calculated with the output p values. C, Heat map displaying correlation matrix of clusters from PBMC samples and TIL. The Pearson correlation coefficient between each RCC PBMC cluster (above the threshold described in the Methods) and each TIL clusters was computed using z-scored values (for RCC PBMC and TIL clusters, respectively, to account for their separate normalization) across all 29 channels shared between each experiment.

FIG. 8A-C. Distribution of T cell phenotypes across tumor types. T cell phenotypes in single cell suspensions of tumors from immunecheckpoint naïve patients were analyzed by mass cytometry and identified using the PhenoGraph algorithm on viable CD45+CD3+ cells. A, Scatter plot indicating the absolute number of CD45+CD3+ live singlets in tumor single cell suspensions from immune checkpoint therapy naïve patients (n=37). Dashed line depicting the 600-cell threshold for sample inclusion (see Methods). B, Box-plots indicating frequencies of selected T cell metaclusters across tumor types (number of patients: NSCLC n=10, RCC n=11, CRC n=9). Samples are identical to samples used in FIGS. 2, 3, 5. C, Histograms depicting expression of immune markers on the respective CD4 and CD8 T cell meta-clusters indicated on the left. Related to FIG. 1F.

FIG. 9A-H. Characterization of Myeloid metaclusters. A, Histograms depicting the expression of immune markers on the respective meta-clusters indicated on the left and in FIG. 2A. B, Contour plots indicating the gating strategy used to manually define myeloid cells phenotypically similar to L8 metacluster identified by PhenoGraph. All cells were gated on CD45+ live cells according to the gating strategy outlined in FIG. 5. C, Box plots indicating manually gated L8 subset frequencies as percentage of CD45+ live cells. (Number of patients, GBM=7, NSCLC=11, RCC=11, pCRC=7, mCRC=4, PCa=5). For pairwise comparisons, p values were computed by Mann-Whitney tests. Q values were calculated with the output p values using the Benjamini-Hochberg method. D, Histogram overlay of CD73 expression of CD68+ cells in normal donor PBMCs (blue) and GBM-TILs (red) by CyTOF. E, Representative IHC images of GBM patient samples F, Box-plot indicating density of CD3+, CD8+ and CD68+ cells/mm² in IHC sections of GBM patients samples (n=7) G, Representative images of multicolor IF in GBM tumor samples (n=6). H, Box-plot indicating percentage of CD68+ cells and CD68+CD73+ cells in total nucleated cells (n=6).

FIG. 10A-B. Similarities of tumor infiltrating leukocyte phenotypes between first and second cohort of untreated GBM patients. Leukocyte phenotypes in single cell suspensions of tumors from immunecheckpoint naïve patients were analyzed by mass cytometry and identified using the PhenoGraph algorithm on viable CD45+ cells. A, Grouped box-plots indicating frequencies of CD45+ cells as indicated in single cell suspension of tumors from untreated cohort 1 patients (n=7) and untreated cohort 2 patients (n=9). Boxes indicate interquartile range with central bar indicating median and whiskers indicating the range. Individual patients are represented with dots. B, Heatmap indicating normalized expression of selected markers on CD45+ meta-clusters identified by PhenoGraph from cohort 2 patients.

FIG. 11A-B. Distribution of tumor infiltrating leukocyte phenotypes in pembrolizumab treated and untreated GBM patients. Leukocyte phenotypes in single cell suspensions of tumors from immunecheckpoint naïve patients (untreated) and Pembrolizumab treated patients (pembro) were analyzed by mass cytometry and identified using the PhenoGraph algorithm on viable CD45+ cells. A, Scatter plot indicating the absolute number of CD45+ live singlets in single cell suspension of tumors from untreated patients (n=8) and pembro treated patients (n=5). B, Grouped box-plots indicating CD45+ immune meta-cluster frequencies identified by PhenoGraph in untreated (n=7) and pembrolizumab treated tumors (n=5).

FIG. 12A-E. Distribution of tumor infiltrating leukocyte phenotypes from orthotopically injected GL-261 gliomas in untreated wild type and CD73^{-/-} mice. CD73^{-/-} and WT mice were inoculated with GL-261 gliomas intracranially. A, Left: Box plots indicating tumor sizes as determined by MRI in WT (blue) and CD73^{-/-} mice (red). Data is representative of two independent experiments, n=5 mice per group. P values were calculated using Mann-Whitney U tests. Boxes indicate interquartile range with central bar indicating median and whiskers indicating the range. Right: Indicated are representative MRI images on day 14 of tumor cell inoculation. Arrows indicate the tumor bulks. B, Kaplan-Meier plot showing overall survival of untreated wild-type or CD73^{-/-} (n=10) orthotopically injected with GL-261 gliomas, P values were calculated using a logrank test (two sided). Data shown are representative of two experiments. C, Representative heatmap indicating intra-tumoral CD11b+ immune populations in both WT and CD73^{-/-} mice bearing GBM tumors by FlowSOM analysis. D, The clusters on the right indicate clusters that show significant changes. P-values were calculated using Mann-Whitney U tests (two sided) and corrected for multiple comparisons using the Benjamini-Hochberg method. Data is representative of two independent experiments, n=5 mice per group. E, Bar graphs depicting CD45+ immune cluster frequencies identified by heatmap (n=5 mice per group). Boxes indicate interquartile range with central bar indicating median and whiskers indicating the range. Individual mice are represented with dots.

### DETAILED DESCRIPTION OF THE INVENTION

Immune checkpoint therapy (ICT) with anti-CTLA-4 and anti-PD-1/PD-L1 has revolutionized the treatment of many solid tumors. However, the clinical efficacy of ICT is limited to a subset of patients with specific tumor types (1,2). Multiple clinical trials with combinatorial immune checkpoint strategies are ongoing, however, the mechanistic rationale for tumor specific targeting of immune checkpoints remains elusive. To garner insight into tumor specific immunomodulatory targets, the inventors analyzed tumors (N=94) representing 5 different cancer types, including those that respond relatively well to ICT and those that do not, such as glioblastoma (GBM), prostate cancer (PCa) and colorectal cancer (CRC). Through mass cytometry and single cell RNA-sequencing, the inventors identified a unique population of CD73hi macrophages in GBM that persists after anti-PD-1 treatment. To test if targeting CD73 would be important for a successful combination strategy in GBM, the inventors performed reverse translational studies using CD73^{-/-} mice. The inventors found that the absence of CD73 improved survival in a murine model of GBM treated with anti-CTLA-4 and anti-PD-1. The data identified CD73 as a specific immunotherapeutic target to improve anti-tumor immune responses to ICT in GBM, and demonstrate that comprehensive human and reverse translational studies can be used for rational design of combinatorial immune checkpoint strategies.

### IV. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### Immune Checkpoint Blockade Therapy

Embodiments of the disclosure may include administration of immune checkpoint blockade therapy, which are further described below.

### PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. patent application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the ICB therapy comprises a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the ICB therapy comprises a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the ICB therapy comprises an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as an ICB therapy in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

B. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

D. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

E. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

F. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### V. Additional Therapies

The current methods and compositions of the disclosure may include one or more additional therapies known in the art and/or described herein. In some embodiments, the additional therapy comprises an additional cancer treatment. Examples of such treatments are described herein, such as the immunotherapies described herein or the additional therapy types described in the following.

### Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

B. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

C. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

D. Chemotherapies

In some embodiments, the additional therapy comprises a chemotherapy. Suitable classes of chemotherapeutic agents include (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozotocin, streptozocin) and triazines (e.g., dacarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophyllotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluorouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2- to 10,000-fold less than the chemotherapeutic effective dose of the chemotherapeutic agent. For example, the chemotherapeutic agent may be administered in an amount that is about 20-fold less, about 500-fold less or even about 5000-fold less than the effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

E. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Grays (Gy) and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

F. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

G. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments, the sample comprises a fractionated sample, such as a blood sample that has been fractionated by centrifugation or other fractionation technique. The sample may be enriched in white blood cells or red blood cells. In some embodiments, the sample may be fractionated or enriched for leukocytes or lymphocytes. In some embodiments, the sample comprises a whole blood sample.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VII. Cancer Monitoring

In certain aspects, the methods of the disclosure may be combined with one or more other cancer diagnosis or screening tests at increased frequency if the patient is determined to be at high risk for recurrence or have a poor prognosis based on the biomarker expression described above, such as expression level and/or presence of CD73 positive macrophages in a biological sample from the subject.

In some embodiments, the methods of the disclosure further include one or more monitoring tests. The monitoring protocol may include any methods known in the art. In particular, the monitoring include obtaining a sample and testing the sample for diagnosis. For example, the monitoring may include endoscopy, biopsy, endoscopic ultrasound, X-ray, barium swallow, a Ct scan, a MRI, a PET scan, laparoscopy, or HER2 testing. In some embodiments, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

### VIII. ROC Analysis

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1—specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### IX. Nucleic Acid Assays

Aspects of the methods include assaying nucleic acids to determine expression or activity levels and/or the presence of CD73 expressing cells in a biological sample. Arrays can be used to detect differences between two samples. Specifically contemplated applications include identifying and/or quantifying differences between RNA from a sample that is normal and from a sample that is not normal, between a cancerous condition and a non-cancerous condition, between one cancerous condition, such as fast doubling time cells and another cancer condition, such as slow doubling time cells, or between two differently treated samples. Also, RNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

To determine expression levels of a biomarker, an array may be used. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

Further assays useful for determining biomarker expression include, but are not limited to, nucleic amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

A further assay useful for quantifying and/or identifying nucleic acids, such as nucleic acids comprising biomarker genes, is RNAseq. RNA-seq (RNA sequencing), also called whole transcriptome shotgun sequencing, uses next-generation sequencing (NGS) to reveal the presence and quantity of RNA in a biological sample at a given moment in time. RNA-Seq is used to analyze the continually changing cellular transcriptome. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

### X. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbent assay (ELISA). A skilled artisan can readily adapt known protein/antibody detection methods for use in determining protein expression levels of biomarkers.

In one embodiment, antibodies, or antibody fragments or derivatives, can be used in methods such as Western blots, ELISA, flow cytometry, or immunofluorescence techniques to detect biomarker expression and/or the presence of cell surface markers such as CD73. In some embodiments, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some embodiments, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 1251, 3H, and 1311. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

### XI. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### XII. Methods of Treatment

Provided herein are methods for treating or delaying progression of cancer in an subject through the administration of therapeutic compositions.

In some embodiments, the therapies result in a sustained response in the individual after cessation of the treatment. The methods described herein may find use in treating conditions where enhanced immunogenicity is desired such as increasing tumor immunogenicity for the treatment of cancer.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

In some embodiments of the methods of the present disclosure, the cancer has low levels of T cell infiltration. In some embodiments, the cancer has no detectable T cell infiltrate. In some embodiments, the cancer is a non-immunogenic cancer (e.g., non-immunogenic colorectal cancer and/or ovarian cancer). Without being bound by theory, the combination treatment may increase T cell (e.g., CD4+ T cell, CD8+ T cell, memory T cell) priming, activation, proliferation, and/or infiltration relative to prior to the administration of the combination.

The cancer may be a solid tumor, metastatic cancer, or non-metastatic cancer. In certain embodiments, the cancer may originate in the bladder, blood, bone, bone marrow, brain, breast, urinary, cervix, esophagus, duodenum, small intestine, large intestine, colon, rectum, anus, gum, head, kidney, liver, lung, nasopharynx, neck, ovary, prostate, skin, stomach, testis, tongue, or uterus.

The cancer may specifically be of the following histological type, though it is not limited to these: neoplasm, malignant; carcinoma; undifferentiated, bladder, blood, bone, brain, breast, urinary, esophageal, thymomas, duodenum, colon, rectal, anal, gum, head, kidney, soft tissue, liver, lung, nasopharynx, neck, ovary, prostate, skin, stomach, testicular, tongue, uterine, thymic, cutaneous squamous-cell, noncolorectal gastrointestinal, colorectal, melanoma, Merkel-cell, renal-cell, cervical, hepatocellular, urothelial, non-small cell lung, head and neck, endometrial, esophagogastric, small-cell lung mesothelioma, ovarian, esophagogastric, glioblastoma, adrenocortical, uveal, pancreatic, germ-cell, giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; cutaneous melanoma, blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises cutaneous squamous-cell carcinoma, non-colorectal and colorectal gastrointestinal cancer, Merkel-cell carcinoma, anal cancer, cervical cancer, hepatocellular cancer, urothelial cancer, melanoma, lung cancer, non-small cell lung cancer, small cell lung cancer, head and neck cancer, kidney cancer, bladder cancer, Hodgkin's lymphoma, pancreatic cancer, or skin cancer.

In some embodiments, the cancer comprises lung cancer, pancreatic cancer, metastatic melanoma, kidney cancer, bladder cancer, head and neck cancer, or Hodgkin's lymphoma.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some embodiments, the treatment may exclude prevention of the disease.

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer or cancer metastasis in patients determined to have a certain gut microbiome composition.

### XIII. Kits

Certain aspects of the present invention also concern kits containing compositions of the invention or compositions to implement methods of the invention. In some embodiments, kits can be used to evaluate expression levels and/or the presence or absence of cell-surface markers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, detection agents, antibodies or inhibitors, or any value or range and combination derivable therein. In some embodiments, there are kits for evaluating expression levels and/or cell surface expression of biomarkers in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for biomarker expression levels.

It is contemplated that any method or composition described herein can be implemented with respect to any other method or composition described herein and that different embodiments may be combined. The claims originally filed are contemplated to cover claims that are multiply dependent on any filed claim or combination of filed claims.

Embodiments of the disclosure include kits for analysis of a pathological sample by assessing biomarker expression profile for a sample comprising, in suitable container means, two or more probes or detection agents, wherein the probes or detection agents detect one or more markers identified herein.

### XIV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Immune Profiling of Human Tumors Identifies CD73 as a Combinatorial Target in Glioblastoma

A. Results

ICT provides durable anti-tumor response to a subset of patients with specific tumor type (3-9). Independent studies have recently provided in-depth single-cell analyses of tumor infiltrating leukocytes (TILs) from individual tumors namely renal cell carcinoma (RCC), hepatocellular carcinoma (HCC), Non-Small Cell Lung Carcinoma (NSCLC) and melanoma (10-13). These studies bring new insights and validate prior findings on the immune infiltrates of different cancers, but the non-uniformity of response amongst cancer types may be a result of tumor type-specific immune checkpoint expression patterns and demands a comprehensive comparison of the TIL phenotypes across multiple tumors. To address this need, the inventors applied mass cytometry (CyTOF) to profile immune cell subsets in 85 patients with 5 different tumor types: NSCLC (n=15), RCC (n=25), MSI stable Colorectal Cancer (CRC) (n=11), Prostate Cancer (PCa) (n=21) as well as Glioblastoma Multiforme (GBM) (n=13) (Supplementary Table 1). This is the first CyTOF dataset evaluating immune cell subsets across different human tumor types.

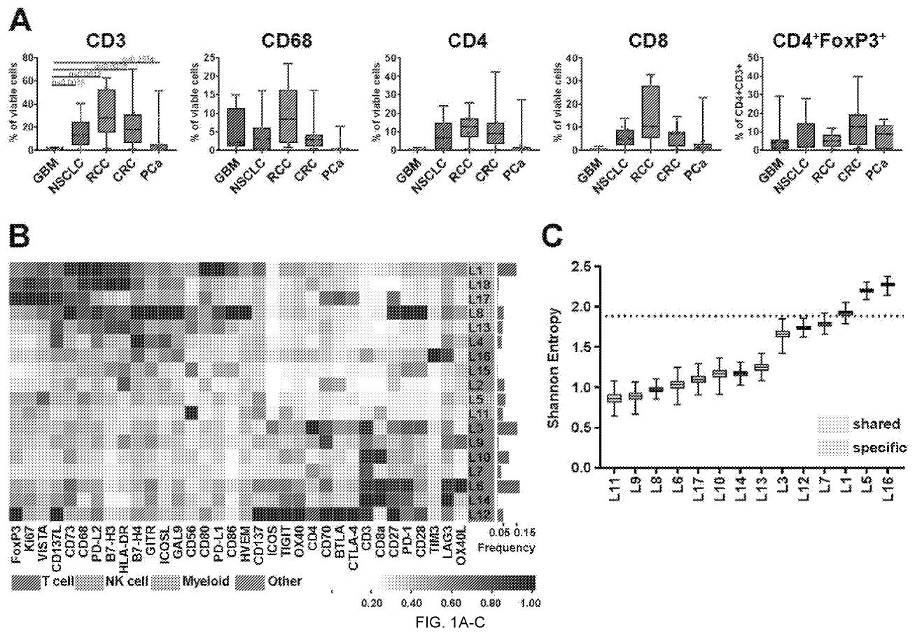

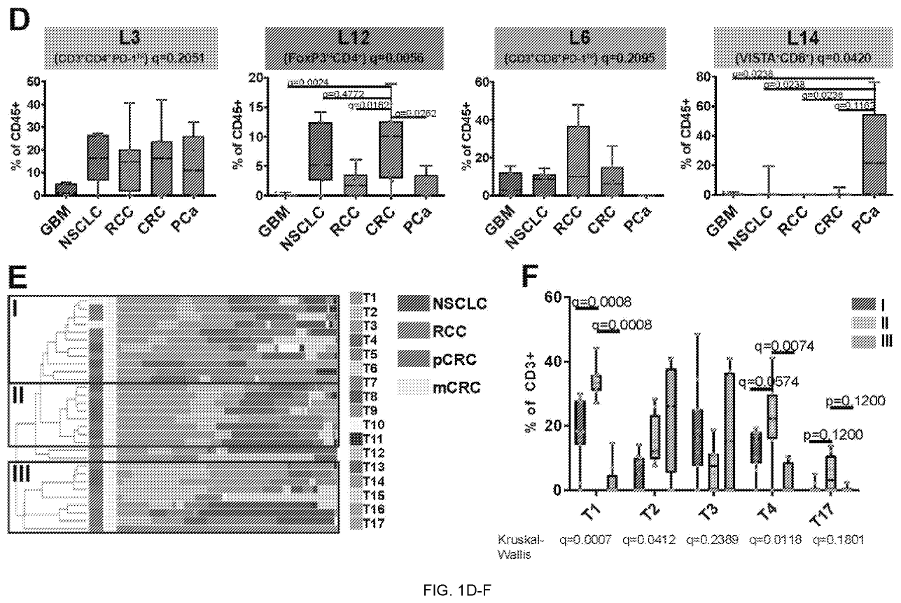

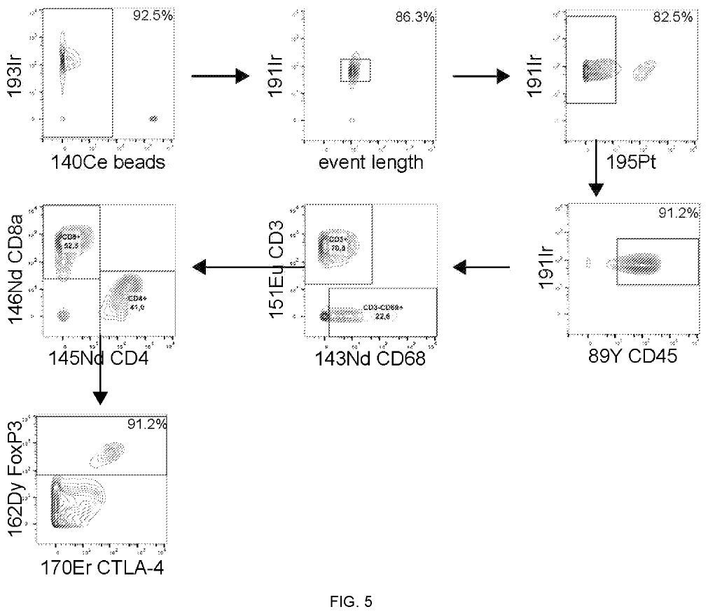

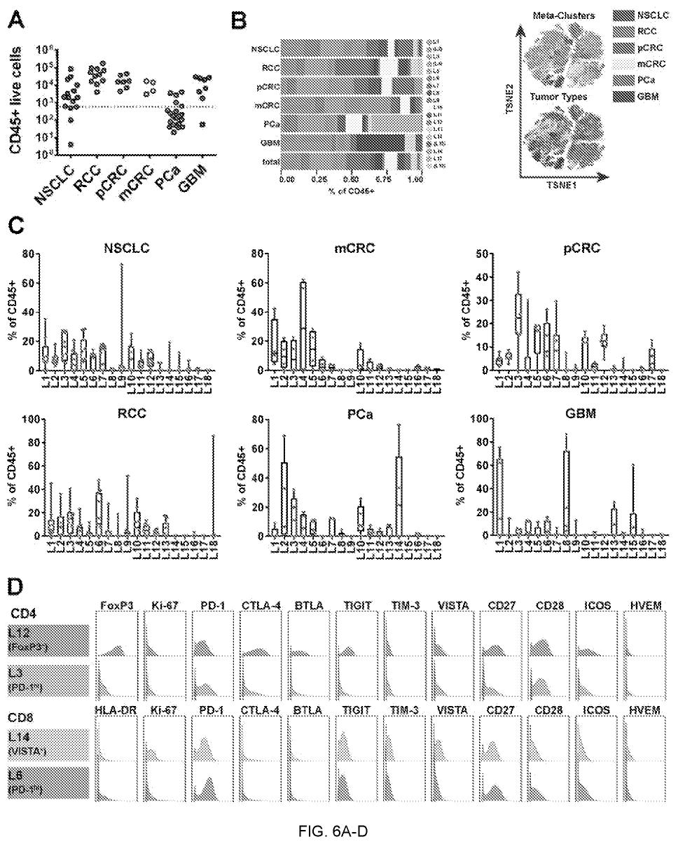

The inventors first compared the major immune infiltrates present in each tumor type (FIG. 5). The inventors observed that NSCLC, RCC and CRC tumors were CD3⁺ T cell rich with CD4⁺FoxP3⁺ cells being most frequent in CRC (FIG. 1A). While both PCa and GBM were poorly infiltrated by CD3⁺ T cells, GBM had higher abundance of CD68⁺ myeloid cells (FIG. 1A). To identify shared phenotypes across the different tumor types, the inventors performed PhenoGraph clustering of CD45+ cells that identified 18 meta-clusters (L1-18), with 8 CD3⁺ T cell meta-clusters and 10 CD3⁻ meta-clusters, including 6 CD68⁺ myeloid clusters and 1 NK cell meta-cluster (FIG. 1B and FIG. 6A-B). The inventors identified a group of 6 immune meta-clusters which were present in all 5 tumor types. These clusters displayed a high Shannon entropy which is a measure of higher uniformity in their distribution across tumor types. The inventors also identified 8 immune meta-clusters that displayed low Shannon entropy values, indicating tumor specific distribution (FIG. 1C).

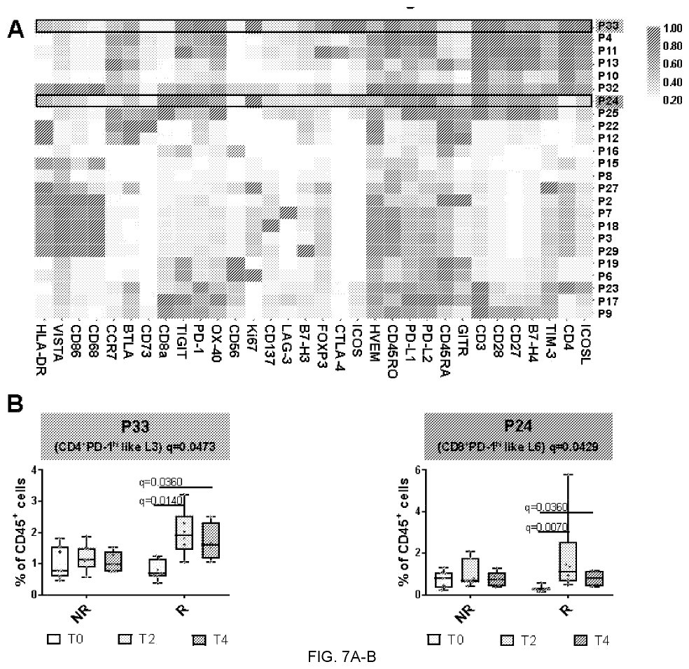

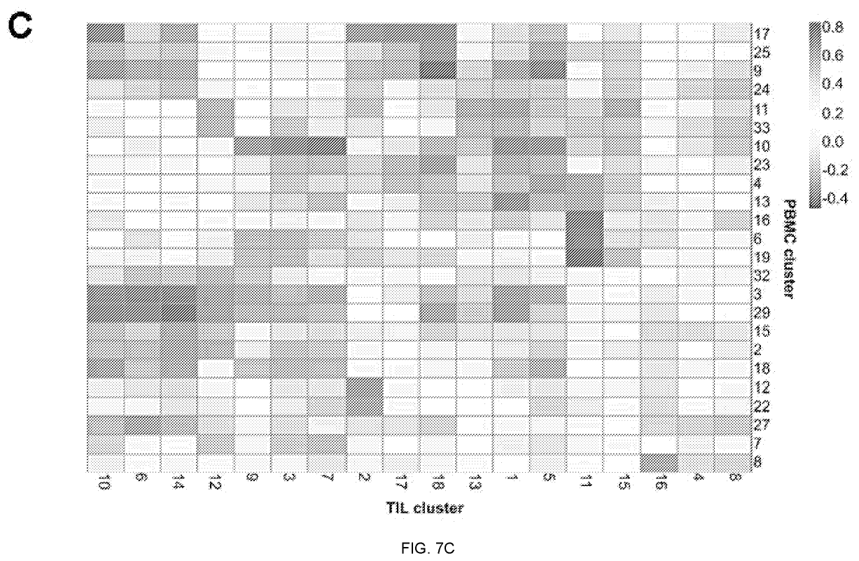

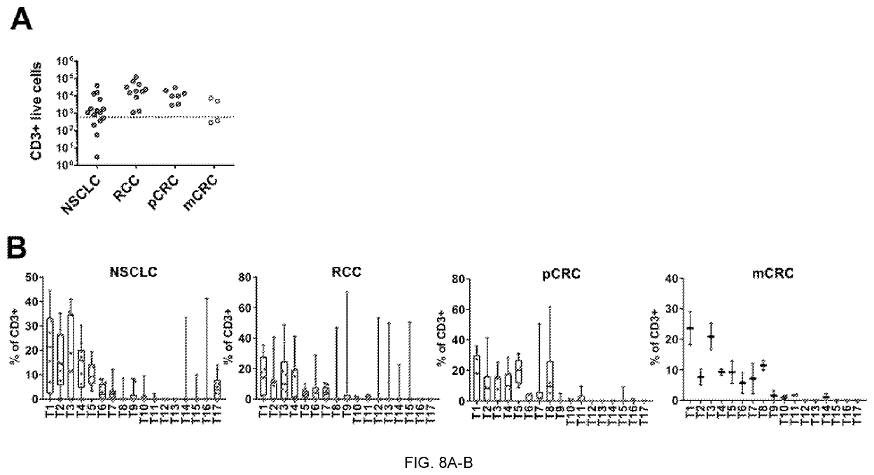

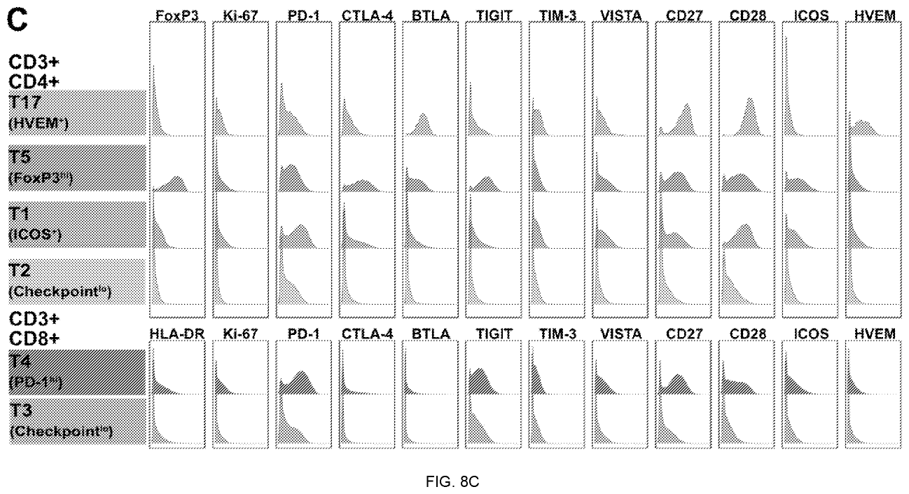

Analysis of the frequency of different T cell clusters identified CD3⁺CD4⁺PD-1^{hi} and CD3⁺CD8⁺ PD-1^{hi} meta-clusters (L3 & L6 respectively) in NSCLC, RCC and CRC (FIG. 1D & FIG. 6C-D). Upon analysis of PBMC samples from the RCC cohort, the inventors identified T cell subsets (P33 and P24) which correlated with L3 and L6 clusters respectively. Interestingly, P33 and P24 clusters were found to be expanded in responders compared to non-responders to ICT (FIG. 7A-C). The inventors also noted higher abundance of CD4⁺FoxP3^{hi} regulatory T cells (L12) and CD8⁺VISTA⁺ (L14) cells in CRC and PCa respectively (FIG. 1D, FIG. 6D), which could be contributing to the lack of response to ICT (14, 15). PhenoGraph clustering of all CD3-gated cells from 30 samples across 3 T cell infiltrated tumor types (NSCLC, RCC and CRC) revealed 17 meta-clusters (FIG. 8A-B). The inventors performed hierarchical clustering of all of these 30 patient samples based on their T cell meta-cluster frequencies and identified 3 primary sub-groups (I, II, and III) (FIG. 1E). A higher frequency of T cell meta-clusters T1 (PD-1^{hi}ICOS⁺CD4⁺ T cell like L3) and T4 (PD-1^{hi} CD8⁺ T cell like L6) were observed in sub-group II, which predominantly comprised NSCLC and RCC, two tumor types that respond favorably to ICT (FIG. 1F). Sub-group III included higher frequencies of meta-clusters T2 (CD4⁺ T cell) and T3 (CD8⁺ T cell), which were low in checkpoint-receptor expression, while sub-group I showed intermediate frequencies of different T cell subsets with both high expression and low expression of immune checkpoints (FIG. 8C).

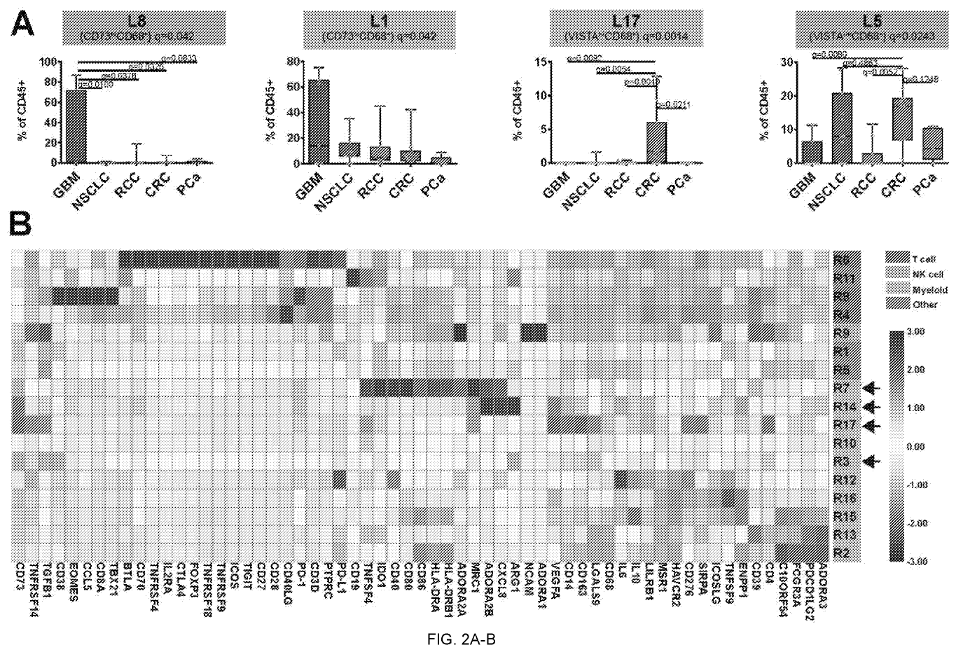

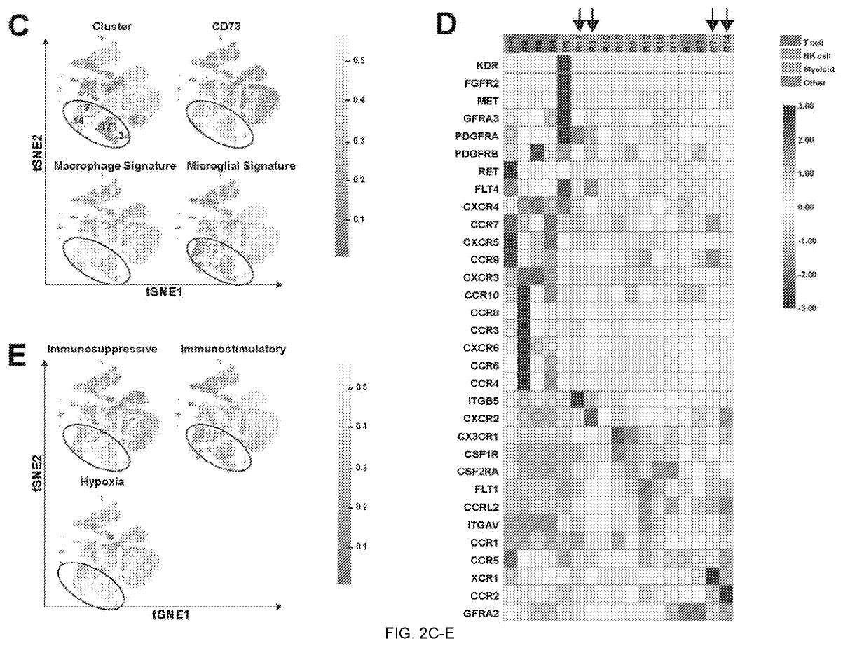

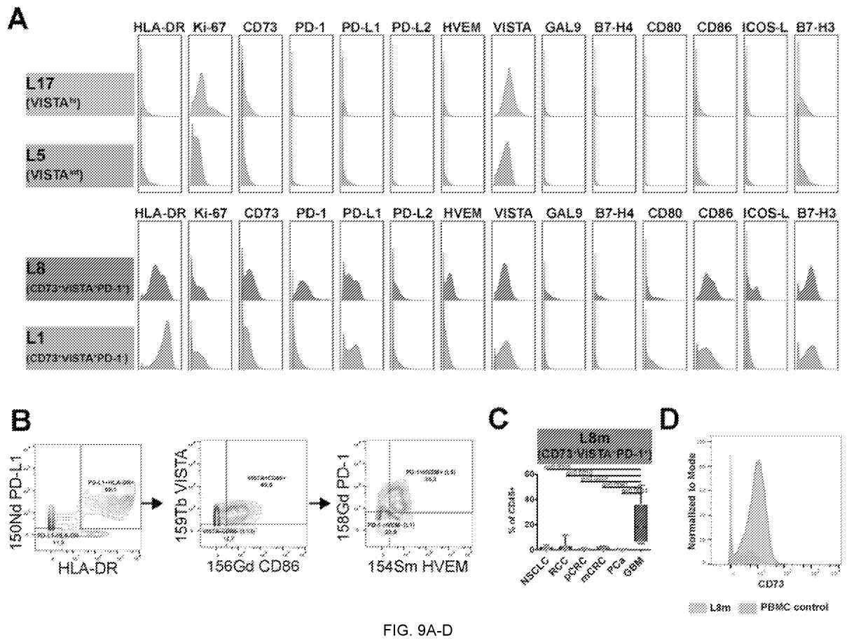

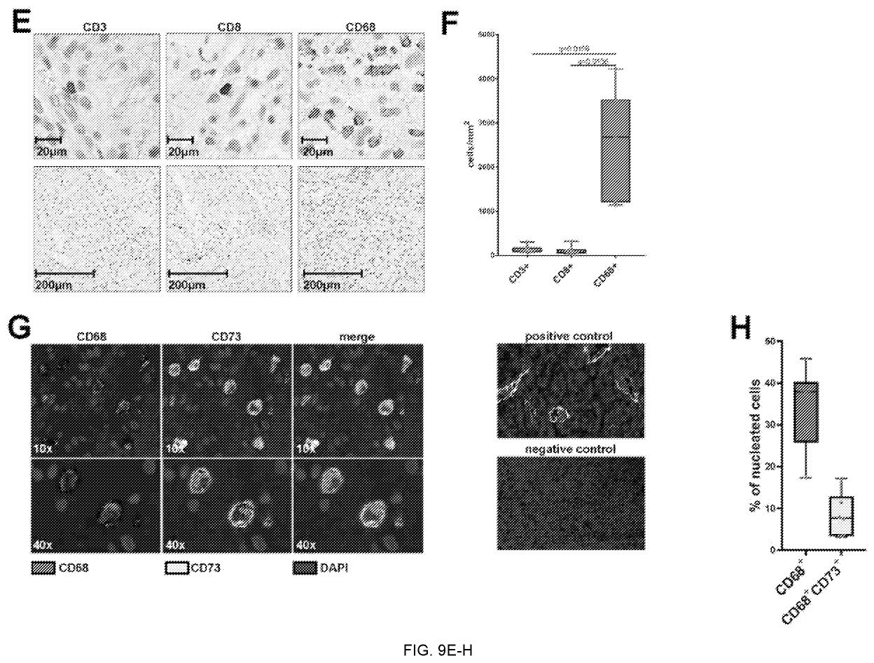

Next, the inventors performed in-depth analysis of the CD3⁻CD68⁺ myeloid clusters identified from the PhenoGraph clustering of CD45⁺ cells across the different tumor types. The inventors observed 2 PD-L1 subset (L5 and L17) and 2 PD-L1⁺ subsets (L1 and L8) across tumor types (FIG. 2A & FIG. 9A). L5 was identified as a VISTA⁺ subset and was present at a higher frequency in CRC as compared to NSCLC and PCa. L17 was also identified as a VISTA⁺ subset but was only found in CRC. L1 was identified as myeloid subset shared by all tumor types.

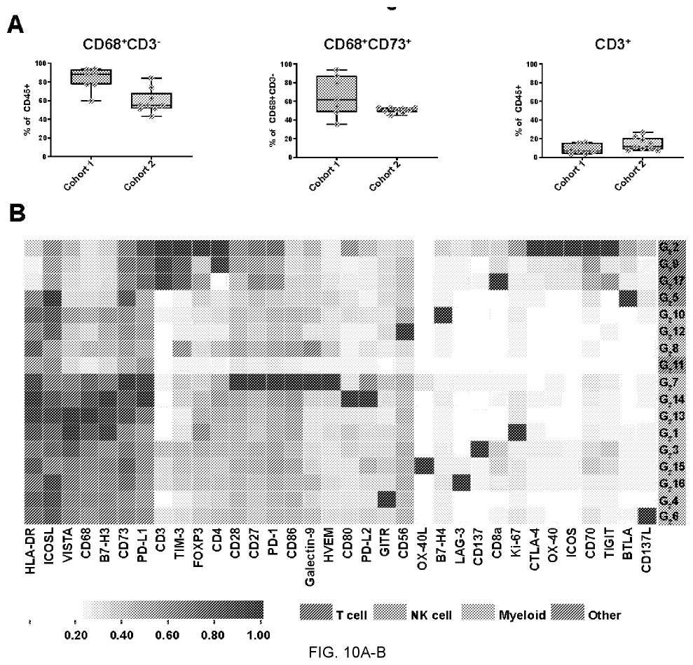

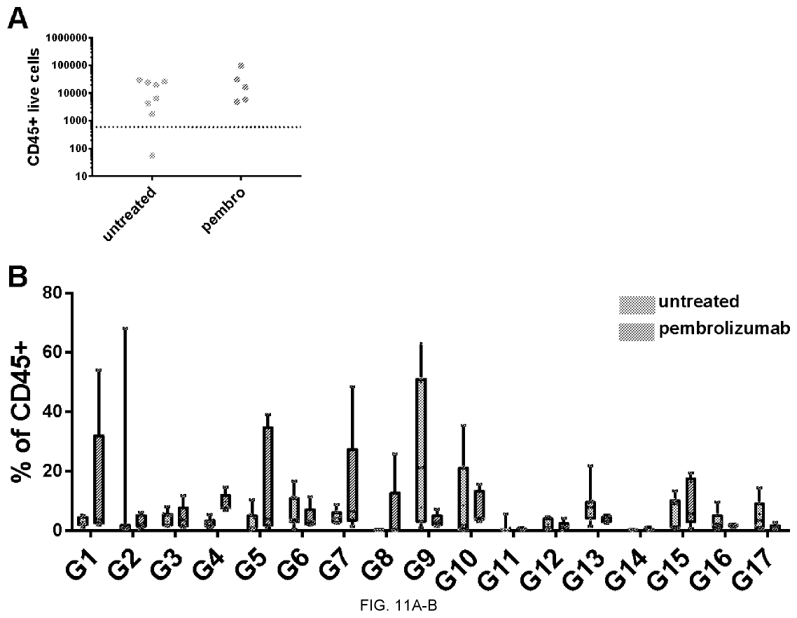

Meta-cluster L8 was a unique subset found only in GBM (this was further validated by manual gating) (FIG. 2A & FIG. 9A-C). L8 expressed high levels of CD73 in addition to other co-inhibitory molecules such as VISTA and PD-1 (FIG. 9D). IHC and IF studies further revealed that human GBM tumors have high density of CD68⁺ macrophages that co-express CD73 (FIG. 9E-H). To demonstrate the validity of these findings on leukocyte infiltration in GBM the inventors analyzed macrophage and T cell infiltration by CyTOF in an independent cohort of 9 GBM patients (FIG. 10). As compared to the first GBM cohort, the inventors found similar high frequencies of CD73^{hi} macrophages and low T cell numbers.

CD73 is an ectonucleotidase which works with its upstream signaling molecule CD39 to convert extracellular ATP to adenosine (16). CD73 has been shown to promote tumor progression and induce immune suppression in GBM (16-20). Further, it was recently shown that kynurenine produced by murine GBM cells can upregulate CD39 in macrophages (19). To obtain a deeper understanding of genes that may define CD73^{hi} myeloid cells the inventors performed single cell RNA sequencing (sc-RNA seq) on 4 additional GBM tumors (Supplementary Table 1). This analysis revealed 17 clusters, of which 4 were CD3⁺ T cell clusters and 10 were CD3⁻CD68⁺ myeloid cell clusters. Of the 10 myeloid clusters, 4 were CD73^{hi} (R7, R14, R3 and R17) (FIG. 2B, indicated by arrows). The inventors found that CD73^{hi} myeloid clusters had high expression of genes suggestive of a blood derived macrophage signature as opposed to microglial signature (21) (FIG. 2C). CD73^{hi} macrophages were also found to express CCR5, CCR2, ITGAV/ITGB5 and CSF1R suggesting that CD73^{hi} macrophages are probably recruited to the GBM tumor microenvironment by these factors (22-26) (FIG. 2D). The inventors also evaluated the CD73^{hi} myeloid cells for expression of immunostimulatory genes or immunosuppressive genes and found that CD73^{hi} myeloid cells had high expression of immunosuppressive and hypoxia related genes (FIG. 2E).

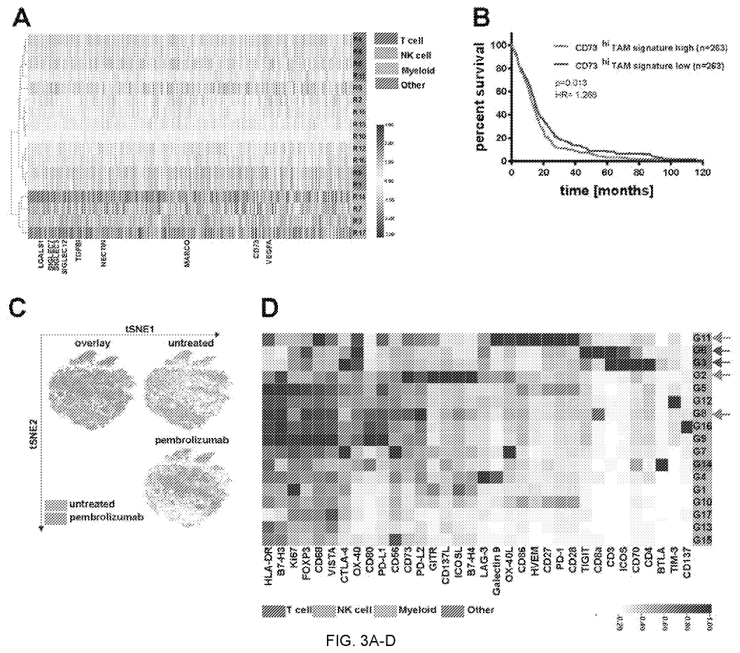

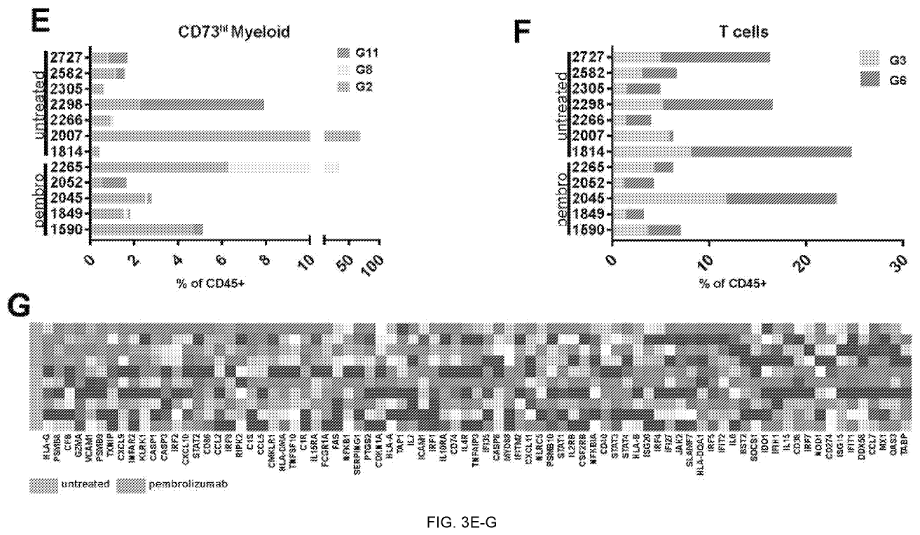

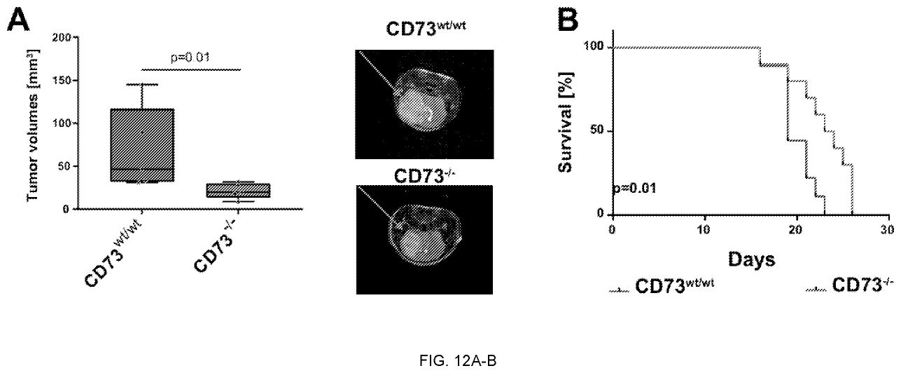

Next, the inventors derived a gene signature specific for CD73^{hi} macrophages using 4 CD73^{hi} clusters (R7, R14, R3 and R17) (FIG. 3; see methods). MARCO, TGFB and several SIGLECs were found to be expressed in the CD73^{hi} cells (FIG. 3A). To understand the significance of the gene signature, the inventors evaluated the CD73^{hi} gene signature for potential correlation with survival. To perform this analysis the inventors used the TCGA-GBM cohort (N=525). The inventors found a significant negative correlation between overall survival (OS) and high expression of the CD73^{hi} gene signature (FIG. 3B, p=0.013, HR=1.268) in TCGA-GBM cohort. Based on the potential immune-suppressive function of CD73^{hi} myeloid cells, the inventors evaluated GBM samples from patients treated with anti-PD-1 to determine whether prevalence of these cells may correlate with lack of response to therapy. The inventors used a cohort of 5 patients with GBM who were enrolled on a phase II study assessing the effect of pembrolizumab in patients with recurrent GBM (NCT02337686, Methods). PhenoGraph clustering of 7 untreated tumors and the cohort of 5 patients with GBM treated with pembrolizumab revealed 17 clusters, consisting of 12 subsets that were characterized as CD3⁻CD68⁺ myeloid subsets, 2 CD3⁺ T cell subsets and 1 NK cell CD3⁻CD56⁺ subset (FIG. 3C-D; FIG. 11). Out of the 12 CD68⁺ myeloid subsets, there were 3 CD73^{hi} myeloid clusters (FIG. 3D; G2, G8, G11 indicated by red arrows). Upon comparison of untreated with anti-PD-1 treated GBM samples, the inventors found that these 3 CD73^{hi} myeloid clusters persisted despite treatment with ICT (FIG. 3E). Evaluation of the remaining myeloid-like clusters that were CD73 low or CD73 negative also persisted despite treatment with ICT, which is consistent with results from a previous study in which there was no change in the myeloid cell markers following anti-PD-1 treatment (27). Of note, 2 T cell clusters were identified (FIG. 3D; G3, G6, indicated by blue arrow), representing CD4 and CD8 respectively, which did not demonstrate any significant difference between untreated and anti-PD-1 treated GBM tumors (FIG. 3F). GSEA analysis from the untreated and anti-PD-1 treated tumors revealed higher expression of IFN-γ responsive genes in anti-PD-1 treated patients (FIG. 3G), in accordance with a recent study which suggested moderate clinical benefit of anti-PD-1 treatment in a neoadjuvant setting (28). These findings suggest that anti-PD-1, despite possibly inducing modest immunological responses in TIL, does not profoundly change the GBM TME, which is characterized by its high content of CD73^{hi} myeloid cells. It is possible that the prevalence of the CD73^{hi} myeloid cells contributed to the lack of T cell infiltration thereby leading to poor clinical outcome.

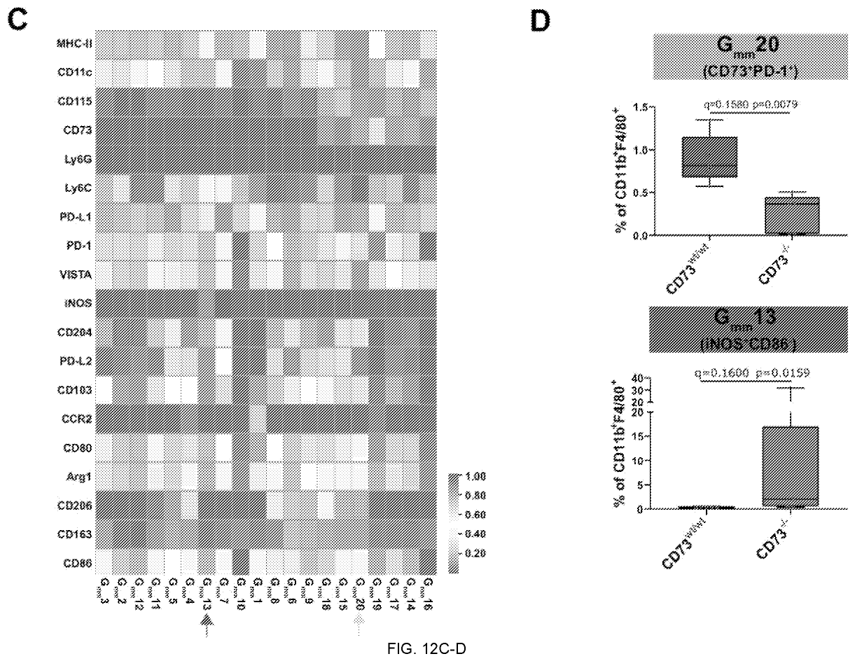

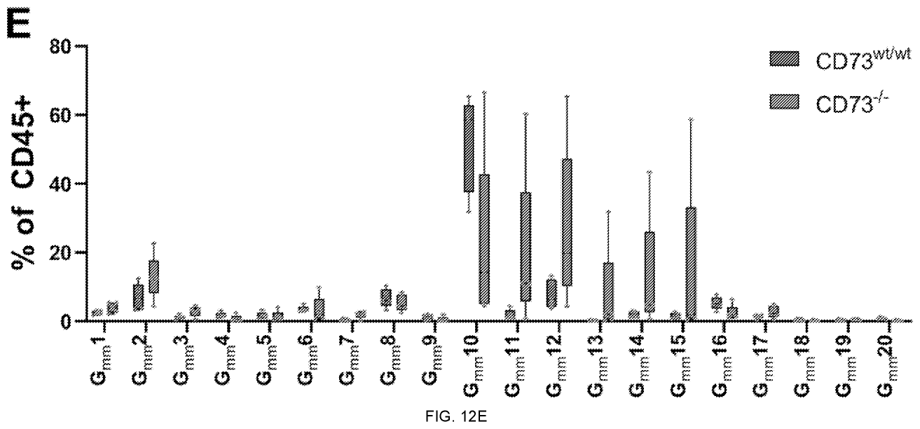

To test the hypothesis that targeting CD73 would be important for a successful combination strategy in GBM, the inventors performed reverse translational studies using wild-type (WT) and CD73^{-/-} mice orthotopically inoculated with GL-261 GBM tumor cells. In the absence of CD73, intracranial tumor growth was impeded (FIG. 12A) and mice exhibited improved survival, confirming the immunosuppressive role of CD73 in GBM (p=0.01) (FIG. 12B). To understand the effect of CD73 in the tumor microenvironment, the inventors performed comparative immune profiling of the tumor microenvironment and assessed the differences in immune infiltrates between the WT and CD73^{-/-} mice using CyTOF (FIG. 12C). Although, the absence of CD73 has been shown to increase intra-tumoral T cell abundance in murine tumor models such as B16-F10 melanoma and MC-38 colon cancer (29), clustering of CD45⁺ gated cells did not reveal significant changes in the T cell subsets between WT and CD73^{-/-} GBM tumor bearing mice. In the GBM model, the inventors noted differences in the myeloid (CD11b⁺F4/80⁺) subsets, including a decrease in the immunosuppressive CD206⁺Arg1⁺VISTA⁺PD-1⁺CD115⁺ myeloid cluster (Gmm20, p=0.0079) in the CD73^{-/-} mice as compared to WT mice (FIG. 12D). Interestingly, the inventors also observed a concomitant increase in iNOS⁺ myeloid clusters (Gmm13, p=0.0159) in the CD73^{-/-} mice (FIG. 12D-E) as compared to the WT mice. This data support the role of CD73 in macrophage polarization. Overall, the data indicate that absence of CD73 in the murine GBM tumor model improves survival by modulating the intra-tumoral myeloid subsets.

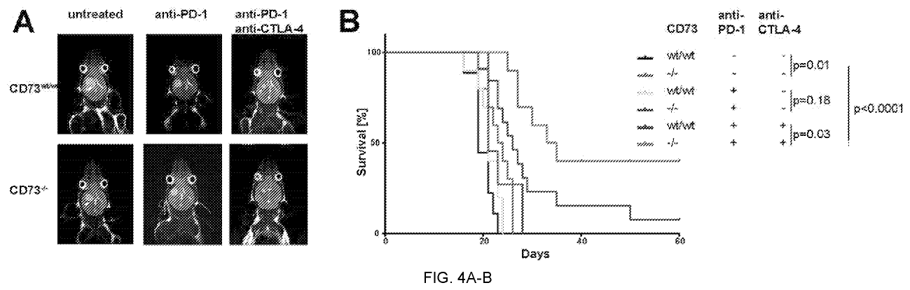

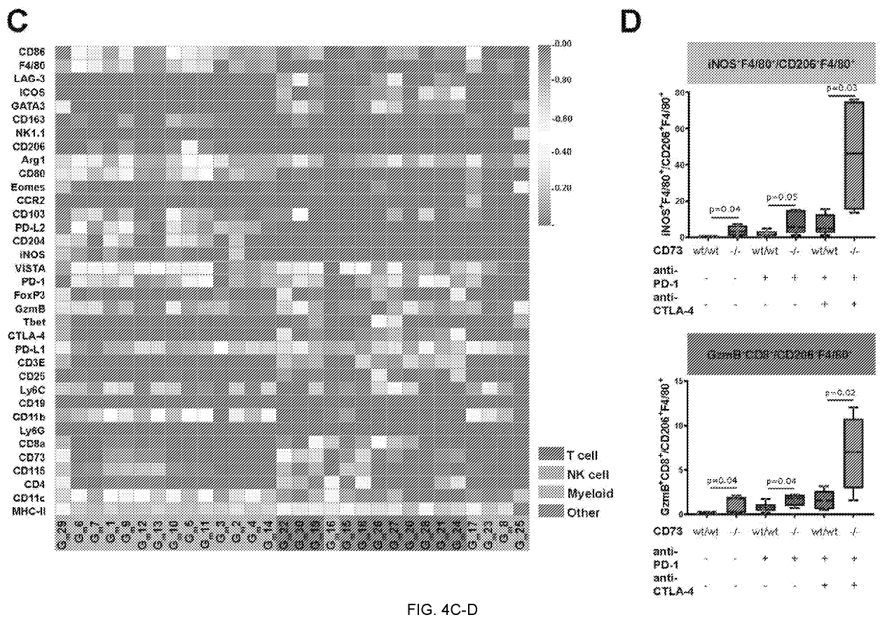

Next, the inventors assessed if CD73-mediated changes in macrophage phenotype could impact the efficacy of ICT. The inventors treated GBM-tumor bearing mice with anti-PD-1 antibody or with a combination of anti-PD-1 and anti-CTLA-4 antibodies. FIG. 4A shows representative MRI images of the GBM tumors from untreated and ICT treated mice. Significant improvement in survival was noted in WT and CD73^{-/-} mice treated with a combination of anti-PD-1 plus anti-CTLA-4 compared to untreated controls (p<0.0001) (FIG. 4B). Importantly, following treatment with combination of anti-PD-1 and anti-CTLA-4, CD73^{-/-} mice had improved survival as compared to WT GBM tumor bearing mice (p=0.03, FIG. 4B). The inventors did not find any significant survival benefit from anti-PD-1 treatment in WT and CD73^{-/-} mice. (FIG. 4B). The inventors noted that the ratio of iNOS⁺ immune stimulatory macrophages to CD206⁺ immune suppressive macrophages was significantly higher in CD73^{-/-} mice compared to WT mice. This was more evident in tumor bearing mice treated with combination therapy. Similarly, the ratio of the granzymeB⁺ CD8 T cells to the CD206⁺ immune suppressive macrophages was significantly higher in the CD73^{-/-} mice compared to WT, and is further pronounced following combination therapy (FIG. 4C-D). These data thus suggest that increased T cell infiltration using combination ICT, coupled with polarization of macrophages to an immune stimulatory phenotype in CD73^{-/-} mice, play a critical part in determining response to ICT.

Multiple immune checkpoints exist (30-32), however, the data suggest dynamic interaction of immune checkpoints in the tumor microenvironment is specific to each tumor type. Clinical trials with combination immunotherapy are ongoing at an unprecedented rate; however, a comprehensive understanding of the tumor-immune interactions are still limited, to design rational combination therapy in a tumor-specific manner. This study coupled in-depth human tumor analyses with murine reverse translational studies to generate a combination strategy for a future clinical trial in GBM. Overall, this study highlights that reverse translational studies are critical to test the relevant hypotheses generated from human datasets for precision immunotherapy.

In this study, the inventors provided immune profiling data from 1) multiple different human tumors and 2) an anti-PD-1 clinical trial in patients with GBM. The inventors identified CD73^{hi} myeloid population to be specifically present in GBM that persisted even after treatment with anti-PD-1 therapy. Further, the inventors derived a gene signature from the CD73^{hi} myeloid cell clusters that negatively correlated with OS in TCGA-GBM cohort. scRNA sequencing showed that CD73^{hi} myeloid cells are enriched in immune-suppressive genes and have a signature distinct from the resident microglial signature. CD73^{hi} myeloid cells are further characterized by higher expression of chemokines/chemokine receptors such as CCR5, CCR2, ITGAV/ITGB5 and CSF1R. Although several clinical trials are testing the utility of targeting these individual chemokine receptors in patients with advanced solid tumors including GBM, cumulative expression of these receptors in CD73^{hi} myeloid cells suggest that CD73 itself is a more relevant target as it is highly expressed on the majority of cells expressing all of these receptors. For example, clinical trials targeting CSF1R have demonstrated limited clinical efficacy (33, 34), which may be due to ongoing presence of myeloid populations expressing other immunosuppressive markers.

This data demonstrate the persistence of an immunosuppressive CD73^{hi} myeloid subsets in patients with GBM who received anti-PD-1 therapy and the therapeutic benefit of immune checkpoint inhibitors in a CD73 deficient mouse model. Based on this data and earlier studies, the inventors propose a combination therapy strategy to target CD73 plus dual blockade of PD-1 and CTLA-4. Anti-CD73 antibody has yielded promising results in preclinical as well as early clinical studies (35, 36), therefore these data have clinical applications with rapid translation of combination therapy for GBM with currently available anti-CD73 antibodies.

B. Methods:

1. Patients and Surgical Samples

Patients with relapsed glioblastoma multiforme were treated with pembrolizumab every 3 weeks on MDACC clinical protocol 2014-0820 (NCT02337686) and consented for PA13-0291. Clinical characteristics of individual patients are indicated in Supplementary Table 1.

2. Cell Lines and Tumor Model

Murine Glioblastoma cancer cell line (GL-261) were obtained from the National Cancer Institute (Rockville, MD, USA). Cells were collected in the logarithmic phase and washed twice with PBS just before tumor injections. 50,000 cells were injected intracerebrally in the mice (5 or 10 mice per group) as described previously (37). Anti-CTLA-4 (clone 9H10) and anti-PD-1 (RMP1-14) antibodies were purchased from BioXcell (West Lebanon, NH). Mice were injected intraperitoneally with anti-PD-1 and combination of anti-PD-1 plus anti-CTLA-4 on day 7 (200 μg/mouse), day 10 (100 μg/mouse) and day 13 (100 μg/mouse) post tumor inoculation.

3. Mass Cytometry (CyTOF)

Patient PBMC were isolated from blood by density gradient centrifugation, resuspended in 90% AB serum and 10% DMSO and stored in liquid nitrogen until the analysis. Fresh tumor tissue was dissociated with GentleMACS system (Miltenyi Biotec; Bergisch Gladbach, Germany) as per the manufacturer's instructions and cultured overnight in a 96 well plate with RPMI 1640 medium; supplemented with 10% human AB Serum, 10 mM Hepes, 50 μM β-ME, penicillin/streptomycin/1-glumacrophagesine. For mouse experiments, freshly collected tumors were dissociated with Liberase/DNAse solution, incubated for 30 minutes at 37° C. prior to single cell being made. Cells were stained with up to 36 antibodies. Antibodies were either purchased pre-conjugated from Fluidigm or purchased purified and conjugated in house using MaxPar X8 Polymer kits (Fluidigm) according to the manufacturer's instructions (See Supplementary Table 4). Briefly, samples were stained with cell-surface antibodies in phosphate-buffered saline (PBS) containing 5% goat serum and 30% BSA for 30 minutes at 4° C. Optimal antibody concentrations were determined by serial dilution stainings of human PBMCs. After viability staining with 5 μM cisplatin (Fluidigm) in PBS containing 30% BSA, samples were washed in PBS containing 30% BSA, fixed and permeabilized according to manufacturers' instructions using the FoxP3 staining buffer set (eBioscience) before being incubated with intracellular antibodies in permeabilization buffer for 30 min at 4° C. Samples were washed and incubated in Ir intercalator (Fluidigm) and stored at 4° C. until acquisition, generally within 12 hours. Right before acquisition samples were washed and resuspended in water containing EQ 4 element beads (Fluidigm). Samples were acquired on a Helios mass cytometer (Fluidigm).

4. Mass Cytometry Analysis

Four separate cohorts of human patient samples were analyzed using CyTOF (after removing samples with too few cells for analysis, as explained for different data sets individually below): 1) 66 samples from TILs extracted from 5 different tumor types; 2) 5 additional GBM TIL samples extracted from tumors resected from patients after treatment with Pembrolizumab; 3) A validation cohort of 9 additional immune checkpoint therapy naïve GBM TIL samples; and 4) 14 Matched PBMC samples from both before and after two and/or four cycles of treatment with combination Ipilimumab and Nivolumab treatment from 14 separate RCC patients. The panels used for the multi-tumor and GBM cohorts were identical (besides one difference as to which channel was used for HLA-DR in some samples, explained below), though a separate panel was used for the RCC PBMC cohort, which was analyzed entirely separately (Supplementary Table 1). For the most part the various analyses using these different cohorts proceeded in a similar if not identical fashion; where they differed will be referenced explicitly below.

First, files (fcs) were uploaded into Cytobank and normalized using a bead-based normalization software for mass Cytometry data (R package premessa, Parker Institute for Cancer Immunotherapy) (Amir el, A. D., et al. viSNE enables visualization of high dimensional single-cell data and reveals phenotypic heterogeneity of leukemia. Nature biotechnology 31, 545-552 (2013)). As the RCC PBMC samples (Cohort 3 above) were labeled using mass-tag cell barcoding for each sample from a given patient, they were additionally demultiplexed using the strategy outlined in Zunder et al., 2015 (Levine, J. H., et al. Data-Driven Phenotypic Dissection of AML Reveals Progenitor-like Cells that Correlate with Prognosis. Cell 162, 184-197 (2015)), prior to bead-based normalization between patients. For the initial TIL (Cohort 1) and additional post-treatment GBM (Cohort 2) samples the inventors merged signals for the 174Yb and 209Bi isotopes into a single channel for HLA-DR, as the inventors used an antibody to HLA-DR that was conjugated to either 174Yb or 209Bi for different samples.

Samples were then manually gated in FlowJo by event length, live/dead discrimination, and for populations of interest using lineage markers (CD45 and CD3) for separate analyses. Data were then exported into Matlab or R as fcs files for downstream analysis, and arcsinh transformed using a coefficient of 5 (x_transformed=arsinh(x/5)). Samples with less than 600 events in the final gate (e.g. CD45⁺ cells or CD3⁺ cells) were excluded due to insufficient cells for clustering, dimension reduction, and other analyses. In the case of the GBM-specific TIL analysis, 4300 cells (chosen as it was the smallest number of viable, post-gating cells from all but one of the samples) were randomly selected from each of 11 samples; as file 1814 contained 1170 cells it was not subsampled and all 1170 cells were included in analysis.

To visualize the high-dimensional data in two dimensions, the t-Distributed Stochastic Neighbor Embedding (t-SNE) dimension reduction algorithm (Van Gassen, S., et al. FlowSOM: Using self-organizing maps for visualization and interpretation of cytometry data. Cytometry A 87, 636-645 (2015)) was applied to the analyses of the multi-tumor TIL samples and separately to the analysis of the 12 total GBM samples (including also 5 post-treatment samples along with the 7 initial samples). For the multi-tumor samples, 10,000 cells were randomly selected from each tumor type, using all markers besides CD326 (EPCAM) and those used to manually gate the population of interest (e.g. CD45 and CD3). For the GBM TIL analysis, subsampling was done as explained above. All t-SNE maps were produced using the Barnes-Hut implementation of the algorithm in the R package Rtsne, and data was displayed using the ggplot2 R package ( ). For t-SNE plots overlaid with expression of individual markers, the arcsinh transformed signal intensity for all values was divided by the 99th percentile of intensity for that channel, leading to signal intensities ranging between 0 and 1 for each channel.

For the murine CyTOF samples, both pre-processing and normalization was done identically (though with an entirely separate murine panel). Clustering and other downstream analysis was done in a different manner, explained below.

5. Mass Cytometry Clustering

Clustering analysis was performed using the MATLAB implementation of the PhenoGraph clustering algorithm (Azizi, E., et al. Single-Cell Map of Diverse Immune Phenotypes in the Breast Tumor Microenvironment. Cell 174, 1293-1308 e1236 (2018)). For the clustering analysis of the multi-tumor samples (Cohort 1), to reduce noise from batch and other effects as well as compress marker redundancies, data from each individual patient were projected onto principal components accounting for 90% of observed variance prior to clustering, using all markers besides CD326 (EPCAM) and those used to manually gate the population of interest (CD45 and CD3, respectively, as well as CD68 for separate T Cell analysis as it was used as a negative gate). This approach was employed to avoid capturing physiologically irrelevant populations as well as reduce residual noise not accounted for by bead normalization. Clusters were identified using PhenoGraph on a per sample basis in the space formed by these principal components, with the parameter k for the number of nearest neighbors selected uniquely for each sample using the formula k=minimum (0.002* number of cells, 10). For each individual sample, pan-positive (expressing high levels of all markers, i.e. likely doublets) and pan-negative (expressing no markers) clusters were excluded from downstream meta-clustering and frequency analyses due to being likely artifacts; they accounted for less than 0.4% of each parent population.

For the murine CyTOF data, normalized data was clustered using the FlowSOM clustering method via Cytobank (van Dijk, D., et al. Recovering Gene Interactions from Single-Cell Data Using Data Diffusion. Cell 174, 716-729 e727 (2018)).

To compare phenotypes across samples while accounting for batch effects, clusters from each sample were represented by their centroid across all non-discarded channels and merged into a single matrix, of size 794 clusters (across 45 samples) by 34 markers for the CD45⁺ TIL analysis, and 486 clusters (across 30 samples) by 32 markers for the CD3⁺ TIL analysis. PhenoGraph was run with parameter k=10 on both of these matrices individually, resulting in 18 meta-clusters in the CD45⁺ analysis, and 17 meta-clusters in the CD3⁺ analysis.

To find tumor type agnostic immune landscape across tumor types, the frequency of cells belonging to each meta-cluster was calculated for each sample and each tumor type in the multi-tumor TIL analysis. Samples were hierarchically clustered by their meta-cluster frequencies using hierarchical clustering with Ward's method and visualized with dendrograms.

In the case of the RCC PBMC analysis (FIG. 7), barcoding reduced the need for an initial sample-specific clustering step followed by meta-clustering; consequently, all cells from all pre and post-treatment samples (without subsampling, resulting in over 1 million total cells) were clustered together. In the case of the clustering analysis of the 12 pre or post-treatment GBM samples (FIG. 3A), clustering was also performed on cells (subsampled identically as in the tSNE section above, with 4300 cells from each samples besides 1170 cells from sample 1814) from all samples together, as the number of clusters obtained from each individual patient in this smaller set (˜200 total) did not allow for stable and robust downstream meta-clustering. This may lead to mildly increased batch effects in this particular analysis, which should be accordingly taken into account in interpretation. In this analysis one small pan-positive cluster of 147 cells (0.3% of the total) was also excluded from downstream analysis. All 9 samples in the GBM validation cohort (Cohort 3) were also clustered together. In all of these analyses PCA pre-processing was done as above.

For heatmap displays of marker expression by either cluster or meta-cluster, depending on the analysis, expression was normalized via dividing by the maximum mean cluster value for each parameter and displayed in R with a custom-made script using the geom_tile function in the ggplot2 package. In all box plots, depicted boxes indicate interquartile range with central bar indicating median and whiskers range.

6. Statistical Analysis

Metacluster and subset frequencies were compared in a two-step approach. First, Kruskal-Wallis tests were performed for the 14 metaclusters from the multi-tumor CyTOF analysis and corrected for multiple comparisons using the Benjamini and Hochberg method. L2, L4, L15 and L18 as well as T12, T13 were removed from multiple comparison corrections as they were either not expressed in the analyzed dataset, expressed by only one patient or of undefined lineage and thus not amenable for comparison. Q-values were calculated using the p.adjust( ) function (R studio Version 1.0.153) and q<0.05 was considered statistically significant. Second, pairwise comparisons were only performed for metaclusters/subsets with statistically significant variation across tumor types using Mann-Whitney tests and corrected for multiple comparisons within the respective clusters using the Benamini and Hochberg method with a q<0.05 considered as statistically significant.

For calculation of ratios of the cell cluster frequencies in the murine experiments (FIG. 4D), 3 CD8 T cell clusters expressing granzymeB were identified (clusters 19, 26 and 27) and their cell frequencies were added. Similarly, 4 iNOS expressing myeloid clusters (clusters 1, 2, 6 and 7) were identified and cell frequencies added. Only 1 CD206 expressing myeloid cluster was observed (cluster 5) and hence was taken individually. The cumulative frequencies of granzyme B⁺CD8 T cell clusters and the cumulative frequencies of iNOS⁺ myeloid clusters were divided by the frequency of CD206⁺ myeloid cluster respectively and the ratios were plotted in GraphPad Prism 7 to obtain statistics. Summary of the statistical methods used for these analyses are included in Supplementary Table 2.

7. Cluster Mixing

Mixing of the 18 CD45⁺ immune meta-clusters across the 6 tumor types (including mCRC) was estimated using a bootstrapping technique to correct for the different sizes of clusters, which ranged from just over 1,800 cells to just over 180,000 cells. Shannon entropy was computed for the empirical distribution of tumor types across 1000 cells, sampled uniformly from each cluster with replacement. This sampling procedure was repeated 1000 times per cluster in order to bootstrap cluster size-corrected standard errors of entropy. FIG. 2C shows boxplots of entropy values in each cluster, ordered by mean entropy.

8. Immunohistochemistry

For IHC analyses, GBM tumor tissues were fixed in 10% formalin, embedded in paraffin, and transversely sectioned. Sections of 4 μm were stained with hematoxylin and eosin (H&E). IHC analyses were conducted on paraffin-embedded tissue sections. Primary antibody was used to detect CD3 (Dako, Cat #A0452), CD8 (Thermo Scientific, Cat #MS-457-S), CD68 (Dako, Cat #M0876). Antibodies were detected with secondary antibodies, followed by peroxidase-conjugated avidin/biotin and 3,3′-diaminobenzidine (DAB) substrate (Leica Microsystem). All IHC slides were scanned and digitalized using the scanscope system from Scanscope XT, Aperio/Leica Technologies. Quantitative analyses of IHC staining were conducted using the image analysis software provided (ImageScope-Aperio/Leica). Five random areas (at least 1 mm2 each) were selected using a customized algorithm for each specific marker for analysis of density of positive cells (numbers of positive cells/mm2).

9. Multiplex Immunofluorescence Assay and Multispectral Analysis

For multiplex staining, the inventors followed the Opal protocol staining method (Finck, R., et al. Normalization of mass cytometry data with bead standards. Cytometry A 83, 483-494 (2013)) for the following markers: CD73 (1:200, Abcam, ab91086) with subsequent visualization using fluorescein Cy3 (1:50); CD163 (1:25, Leica Biosystems, NCL-L-CD163) with visualization accomplished using Cy5 (1:50); and CD68 (1:100, Dako, M0876) with visualization using Cy5.5 (1:50). Nuclei were subsequently visualized with DAPI (1:2000). All of the sections were cover-slipped using Vectashield H-1400 mounting media. For multispectral analysis, a detailed methodology was followed as described previously (Stack et al., 2014). Each of the individually stained sections was utilized to establish the spectral library of fluorophores required for multispectral analysis. The slides were scanned using the Vectra slide scanner (PerkinElmer) under fluorescent conditions. For each marker, the mean fluorescent intensity per case was then determined as a base point from which positive cells could be established. Finally, the co-localization algorithm was used to determine percent of CD68, CD163 and CD73 staining.

10. Single-Cell RNA Sequencing

Single-cell RNA sequencing (sc-RNA seq) was performed using the 10× genomics chromium single cell controller. Briefly, tumor cell single cell suspensions were prepared as indicated above. Cells were resuspended in freezing media containing 90% AB serum and 10% DMSO and stored in liquid nitrogen until analysis. For sc-RNA seq analysis cells were thawed, washed and sorted for viable CD45⁺ cells using the BD FACSAria. Next, cells were droplet separated using Chromium™ Single Cell 3′ v2 Reagent Kit with the 10× genomics microfluidic system creating cDNA library with individual barcodes for individual cells. Barcoded cDNA transcripts from GBM patients were pooled and sequenced using the Ilumina HiSeq 4000 Sequencing System.

11. Single Cell RNA Sequencing Clustering and Statistical Analysis

For each of the 4 GBM sc-RNA seq samples Illumina fastq files were preprocessed and converted into count matrices using the Sequence Quality Control (SEQC) package. Briefly, SEQC takes as input Illumina barcode and genomic sequence fastq or bcl files; merges them into a single fastq file containing alignable sequence and metadata; filters reads for common errors including barcode substitution errors and low-complexity errors; aligns reads using STAR; resolves multiple alignment reads; and groups the error-reduced and filtered reads by cell, molecule, and gene annotation into count matrices. It also outputs a series of QC metrics by which to evaluate the library quality. The pipeline is described in full detail in Azizi, et al. 2018.

These four separate count matrices were then merged into one large count matrix, of size 13,263 cells (ranging from 2,763 to 3,666 cells per patient) by 19,187 genes. The data was first preprocessed in three sequential ways: first, it was normalized according to the median library—size for each cell, as is the standard practice for sc-RNA seq data; next, it was log-transformed; and finally, principal component analysis was applied to further decrease noise and maximize signal robustness while taking advantage of the redundancy inherent to gene expression (so-called “intrinsic dimensionality”), with principal components accounting for 90% of the variance retained.

Next, the median number of unique molecules (UMI) per cell was low across the four samples (1170, 1210, 1468, and 1592, respectively), resulting in a sparse data matrix, as is common to sc-RNA seq data. Thus, the inventors used the imputation algorithm Markov affinity-based graph imputation of cells (MAGIC) to denoise the count matrix and correct for data sparsity and gene dropout. MAGIC exploits shared information across similar (“neighboring”) cells, via data diffusion, to both de-noise the count matrix and, crucially, fill in missing transcripts that are likely present but have been lost to sampling error (“dropout” or false negatives). This is particularly important in the case of interrogating gene-gene relationships, such as in the case of co-expression patterns in important cell populations. Of minor note, MAGIC also performs PCA as a pre-processing step but returns a full (non-dimension reduced) imputed count matrix; for downstream analysis (clustering, etc.) PCA pre-processing as described above was applied to this imputed count matrix. A full, detailed description of the intuition, biological and mathematical theory, and algorithmic procedure of MAGIC is provided in van Djik et al., 2018. For this analysis, the R implementation of MAGIC was used, with the following parameter settings: all genes; k (number of nearest neighbors) of 10; alpha of 15; and the automatic (“t=auto”) value for the power by which the diffusion operator is powered, such that t was selected according to the Procrustes disparity of the diffused data (the value of t chosen in this manner was 8).

t-SNE visualization of the sc-RNA seq data was again performed using the reduced PCA space, applied to all cells from all four patients, using the Barnes-Hut implementation of the algorithm and signal intensities relative to maximum imputed expression of either the individual markers or the mean expression of multi-gene signatures.

Clustering of the sc-RNA seq data was performed using PhenoGraph in the reduced PCA space on all cells, with k again set to 0.002* number (cells)=38. One cluster of cells, totaling less than one-half of one percent of the total, was identified that did not express any canonical immune typing markers at an appreciable frequency (CD45, CD3, CD8, CD4, CD14, CD68, etc.) It did, however, express high levels of several markers associated with neurons. Thus, the inventors conclude that it is likely a rare contaminant that was erroneously missed by the CD45-based sorting process, and was removed from all analyses and it was outside the scope of the immune populations that are the object of investigation in this study.

Hypoxia, anti-inflammatory (“immunosuppressive”), and pro-inflammatory (“immunostimulatory”) gene signatures were taken from Azizi, et al. 2018, while the microglial versus bone-marrow derived signatures were taken from Muller et al., 2017. In all cases the intensity of expression of the signature in question was computed as the mean expression of the genes included in the signature.

In order to define a gene signature representative of the CD73⁺ macrophage populations of special interest in this study, the four sc-RNA seq PhenoGraph clusters expressing high levels of CD73 and varying combinations of other immuno-suppressive factors (R3, R7, R14, and R17) were grouped into one (with all cells from the four clusters merged), and their differential expression compared to all cells not in one of those four clusters (i.e. belonging to any of the 13 other clusters, including T cell, myeloid, and NK cell populations). Together there were 3453 cells in one of these three clusters. Though traditional bulk RNA-seq methods for differential expression rely on mean expression and fold-change between samples/cell populations, a crucial aspect of single cell data is the ability to utilize the full distribution (with respect to multi-dimensional gene expression) of cells in a population of cells (i.e. a distribution as opposed to a point representation). A method for assessing differential expression between populations that maximally exploits these full distributions, and has been increasingly used in recent studies, is the Earth Mover's Distance (EMD). In physical terms, the EMD quantifies the minimum “cost” of converting one pile of some material (e.g. dirt) into another, defined as the amount of material moved multiplied by the distance by which it is moved. In probability theory, it thus measures the distance between two distributions (again, as opposed to the distance between simply e.g. their means). For one-dimensional distributions (in the case for the distribution of expression of a single gene in a group of cells) it can be conveniently and efficiently computed as the L1 norm of the cumulative density functions for two distributions. Thus, the inventors calculated the EMD using this method for each gene, between the two distributions of interest (cells belonging to the 4 CD73⁺ clusters and cells belonging to all other clusters), and ranked all of the over 19,000 genes by their EMD (with the top genes being differentially highly expressed in the CD73⁺ clusters, and the bottom genes the inverse). The EMD values, and associated z-scores across all genes, are provided for all genes in Supplementary Table 3. All genes with a z-score above 2.0 are shown in FIG. 3A.

12. Nanostring Gene Expression Analysis

RNA were isolated from formalin fixed paraffin embedded (FFPE) tumor sections by de-waxing using deparaffinization solution (Qiagen, Valencia, CA), and total RNA was extracted using the RecoverALL™ Total Nucleic Acid Isolation kit (Ambion, Austin, TX) according to the manufacturer's instructions. The RNA purity was assessed on the ND-Nanodrop1000 spectrometer (Thermo Scientific, Wilmington, MA, USA). For the NanoString platform, 100 ng of RNA was used to detect immune gene expression using nCounter PanCancer Immune Profiling panel along with custom CodeSet. Counts of the reporter probes were tabulated for each sample by the nCounter Digital Analyzer and raw data output was imported into nSolver (available on the world wide web at nanostring.com/products/nSolver). nSolver data analysis package was used for normalization and hierarchical clustering heatmap analysis were performed with Qlucore Omics Explorer version 3.5 software (Qlucore, NY, USA).

13. MRI Image Quantification

The MRI images were quantified using ImageJ Software version 1.52a. First, the images were imported and the Brightness/Contrast was adjusted. The images slices were then scanned to identify tumor sections. A gate was drawn around the tumor in each section and the area was measured. The image geometry indicated the slice thickness to be 0.75 mm and the distance between two sections to be 1 mm. Tumor area in each section was multiplied by 0.75 and the average between the tumor area in 2 sections was taken and multiplied by (1-0.75) 0.25 (this gave the value for depth). The volume for each tumor was obtained by multiplying the tumor area and depth from section containing tumor. All the values were added to determine the volume of tumor in cubic mm.

14. Survival Analysis

A gene expression signature using this method was defined by taking the top 44 genes, with a z-score above 3.0. The gene expression data based on microarray panel were downloaded from cBioportal (available on the world wide web at cbioportal.org/datasets, Glioblastoma Multiforme (TCGA, Provisional), as of Nov. 7, 2018). In the analysis, the inventors used 525 patients with primary tumors whose clinical data are available. In the provisional dataset the inventors utilized data from 201 patients published in Nature 2008, and the inventors utilized data from 151 patients published in Cell 2013; 35 of the 44 signature genes were used, because 9 genes were not found in the U133 microarray data. The patients were sorted by the average z-score values of the signature genes and then split into a group with high expression (n=263) and a group with low expression (n=262). A log-rank test showed a significant negative association of the survival with the expression level of the signature genes (p=0.013) (FIG. 3B).

15. Statistical Analyses for Murine Experiments:

All data are representative of at least two to three independent experiments with 5-10 mice in each in vivo experiment. The data are expressed as mean±standard error of the mean (SEM) and were analyzed using Prism 7.0 statistical analysis software (GraphPad Software, La Jolla, CA). Student t-tests (two tailed), ANOVA, and Bonferroni multiple comparison tests were used to identify significant differences (p<0.05) between treatment groups. The log-rank test was used to analyze data from the survival experiments.

C. Tables

Supplementary Table 1: Patient characteristics with correlative assays performed

| | Tumor | Primary/ | | | | | | | | | | |
|---|---|---|---|---|---|---|---|---|---|---|---|---|
| Patient # | Type | Metastasis | Pathology | AJCC | Gender | Age | ICT | RT | CT | TT | HT | Panel |
| 1 | NSCLC | primary | SCC | IIIA | m | 59 | n | n | n | n | N/A | CyTOF |
| 2 | NSCLC | primary | AC | IA | f | 63 | n | n | n | n | N/A | CyTOF |
| 3 | NSCLC | primary | AC | IIA | f | 64 | n | y | n | y | N/A | CyTOF |
| 4 | NSCLC | primary | AC | IIIA | f | 64 | n | n | n | n | N/A | CyTOF |
| 5 | NSCLC | primary | AC | IB | f | 69 | n | n | n | n | N/A | CyTOF |
| 6 | NSCLC | primary | AC | IV | m | 54 | n | y | n | n | N/A | CyTOF |
| 7 | NSCLC | primary | AC | IB | m | 66 | n | n | n | n | N/A | CyTOF |
| 8 | NSCLC | primary | AC | IB | f | 73 | n | n | n | n | N/A | CyTOF |
| 9 | NSCLC | primary | SCC | IB | f | 68 | n | n | n | n | N/A | CyTOF |
| 10 | NSCLC | primary | SCC | IIIA | f | 70 | n | n | n | n | N/A | CyTOF |
| 11 | NSCLC | primary | AC | IB | f | 70 | n | y | n | n | N/A | CyTOF |
| 12 | NSCLC | primary | SCC | IIB | m | 57 | n | y | y | y | N/A | CyTOF |
| 13 | NSCLC | primary | AC | IIIA | m | 41 | n | n | n | n | N/A | CyTOF |
| 14 | NSCLC | primary | AC | IIIA | f | 54 | n | n | y | n | N/A | CyTOF |
| 15 | NSCLC | primary | AC | IA | m | 58 | n | n | y | n | N/A | CyTOF |
| 16 | RCC | primary | CC | III | f | 69 | n | n | n | n | N/A | CyTOF |
| 17 | RCC | primary | CC | III | f | 57 | n | n | n | n | N/A | CyTOF |
| 18 | RCC | primary | CP | II | m | 39 | n | n | n | n | N/A | CyTOF |
| 19 | RCC | primary | TC | III | m | 87 | n | n | n | n | N/A | CyTOF |
| 20 | RCC | primary | CC | I | m | 67 | n | n | n | n | N/A | CyTOF |
| 21 | RCC | primary | CC | I | m | 63 | n | n | n | n | N/A | CyTOF |
| 22 | RCC | primary | CC | I | m | 54 | n | n | n | n | N/A | CyTOF |
| 23 | RCC | primary | CC | III | m | 48 | n | n | n | n | N/A | CyTOF |
| 24 | RCC | primary | CC | III | m | 58 | n | n | n | n | N/A | CyTOF |
| 25 | RCC | primary | CC | III | m | 47 | n | n | n | n | N/A | CyTOF |
| 26 | RCC | primary | CC | III | f | 65 | n | n | n | n | N/A | CyTOF |
| 27 | RCC | primary | CC | III | m | 67 | y | n | n | n | N/A | CyTOF |
| 28 | RCC | primary | CC | III | m | 54 | y | n | n | n | N/A | CyTOF |
| 29 | RCC | primary | CC | IV | m | 63 | y | n | n | n | N/A | CyTOF |
| 30 | RCC | primary | CC | IV | m | 47 | y | n | n | n | N/A | CyTOF |
| 31 | RCC | primary | CC | III | m | 62 | y | n | n | n | N/A | CyTOF |
| 32 | RCC | primary | CC | IV | f | 63 | y | n | n | n | N/A | CyTOF |
| 33 | RCC | primary | CC | III | f | 64 | y | n | n | n | N/A | CyTOF |
| 34 | RCC | metastasis | CC | IV | f | 70 | y | n | n | n | N/A | CyTOF |
| 35 | RCC | primary | CC | III | m | 41 | y | n | n | n | N/A | CyTOF |
| 36 | RCC | primary | CC | IV | f | 65 | y | n | n | n | N/A | CyTOF |
| 37 | RCC | primary | CC | IV | m | 59 | y | n | n | n | N/A | CyTOF |
| 38 | RCC | primary | CC | IV | m | 71 | y | n | n | n | N/A | CyTOF |
| 39 | RCC | primary | CC | IV | m | 42 | y | n | n | n | N/A | CyTOF |
| 40 | RCC | primary | CC | III | m | 68 | y | n | n | n | N/A | CyTOF |
| 41 | CRC | primary | MSS | IV | m | 58 | n | y | n | y | N/A | CyTOF |
| 42 | CRC | primary | MSS | IIA | f | 50 | n | n | n | n | N/A | CyTOF |
| 43 | CRC | primary | MSS | IV | m | 44 | n | y | n | y | N/A | CyTOF |
| 44 | CRC | primary | MSS | IIIB | m | 47 | n | n | y | n | N/A | CyTOF |
| 45 | CRC | primary | MSS | IIA | f | 52 | n | n | y | n | N/A | CyTOF |
| 46 | CRC | primary | MSS | III | m | 53 | n | y | y | n | N/A | CyTOF |
| 47 | CRC | primary | MSS | IIIB | m | 65 | n | n | y | n | N/A | CyTOF |
| 48 | CRC | metastasis | unknown | IV | m | 77 | n | y | n | y | N/A | CyTOF |
| 49 | CRC | metastasis | MSS | IV | m | 57 | n | y | y | y | N/A | CyTOF |
| 50 | CRC | metastasis | MSS | IV | f | 54 | n | y | n | y | N/A | CyTOF |
| 51 | CRC | metastasis | MSS | IV | f | 76 | n | y | n | n | N/A | CyTOF |
| 52 | Prostate | primary | Grade II | IIB | m | 63 | n | n | n | n | n | CyTOF |
| 53 | Prostate | primary | Grade V | IV | m | 65 | n | n | n | n | n | CyTOF |
| 54 | Prostate | primary | Grade III | IIIB | m | 68 | n | n | n | n | n | CyTOF |
| 55 | Prostate | primary | Grade V | IIIC | m | 65 | n | n | n | n | y | CyTOF |
| 56 | Prostate | primary | Grade II | IIIB | m | 72 | n | n | n | n | n | CyTOF |
| 57 | Prostate | primary | Grade II | IIB | m | 55 | n | n | n | n | y | CyTOF |
| 58 | Prostate | primary | Grade II | IIB | m | 76 | n | n | n | n | n | CyTOF |
| 59 | Prostate | primary | Grade II | IIA | m | 62 | n | n | n | n | n | CyTOF |
| 60 | Prostate | primary | Grade II | IIB | m | 44 | n | n | n | n | n | CyTOF |
| 61 | Prostate | primary | Grade III | IIC | m | 74 | n | n | n | n | n | CyTOF |
| 62 | Prostate | primary | Grade III | IIIB | m | 62 | n | n | n | n | n | CyTOF |
| 63 | Prostate | primary | Grade III | IIC | m | 51 | n | n | n | n | y | CyTOF |
| 64 | Prostate | primary | Grade II | IIB | m | 70 | n | n | n | n | n | CyTOF |
| 65 | Prostate | primary | Grade II | IIB | m | 66 | n | n | n | n | n | CyTOF |
| 66 | Prostate | primary | Grade II | IIB | m | 54 | n | n | n | n | n | CyTOF |
| 67 | Prostate | primary | Grade II | IIB | m | 54 | n | n | n | n | n | CyTOF |
| 68 | Prostate | primary | Grade V | IIIC | m | 66 | n | n | n | n | y | CyTOF |
| 69 | Prostate | primary | Grade II | IIB | m | 45 | n | n | n | n | n | CyTOF |
| 70 | Prostate | primary | Grade II | IIIB | m | 65 | n | n | n | n | n | CyTOF |
| 71 | Prostate | primary | Grade II | IIB | m | 56 | n | n | n | n | n | CyTOF |
| 72 | Prostate | primary | Grade II | IIB | m | 54 | n | n | n | n | n | CyTOF |
| 73 | GBM | primary | GBM | IV | m | 61 | n | n | n | n | N/A | CyTOF |
| 74 | GBM | primary | GBM | IV | f | 43 | n | n | n | n | N/A | CyTOF |
| 75 | GBM | primary | GBM | IV | m | 45 | n | y | y | y | N/A | CyTOF |
| 76 | GBM | primary | GBM | IV | m | 53 | n | y | y | y | N/A | CyTOF |
| 77 | GBM | primary | GBM | IV | m | 48 | n | y | y | n | N/A | CyTOF |
| 78 | GBM | primary | GBM | IV | m | 59 | n | n | y | n | N/A | CyTOF |
| 79 | GBM | primary | GBM | IV | m | 63 | n | y | y | y | N/A | CyTOF |
| 80 | GBM | primary | GBM | IV | m | 68 | n | y | y | n | N/A | CyTOF |
| 81 | GBM | primary | GBM | IV | f | 45 | y | y | y | n | N/A | CyTOF |
| 82 | GBM | primary | GBM | IV | m | 51 | y | y | y | n | N/A | CyTOF |
| 83 | GBM | primary | GBM | IV | m | 69 | y | y | y | n | N/A | CyTOF |
| 84 | GBM | primary | GBM | IV | f | 69 | y | y | y | n | N/A | CyTOF |
| 85 | GBM | primary | GBM | IV | f | 65 | y | y | y | n | N/A | CyTOF |
| 86 | GBM | primary | GBM | IV | m | 65 | n | y | y | n | N/A | seq |
| 87 | GBM | primary | GBM | IV | m | 36 | n | n | n | n | N/A | seq |
| 88 | GBM | primary | GBM | IV | f | 44 | n | y | y | n | N/A | seq/CyTOF |
| 89 | GBM | primary | GBM | IV | f | 61 | n | y | y | n | N/A | seq |
| 90 | GBM | primary | GBM | IV | m | 62 | n | y | y | y | N/A | CyTOF |
| 91 | GBM | primary | GBM | IV | m | 58 | n | y | y | y | N/A | CyTOF |
| 92 | GBM | primary | GBM | IV | f | 62 | n | y | y | y | N/A | CyTOF |
| 93 | GBM | primary | GBM | IV | m | 65 | n | y | n | n | N/A | CyTOF |
| 94 | GBM | primary | GBM | IV | f | 46 | n | y | y | n | N/A | CyTOF |
| 95 | GBM | primary | GBM | IV | m | 72 | n | y | y | n | N/A | CyTOF |
| 96 | GBM | primary | GBM | IV | m | 23 | n | y | y | n | N/A | CyTOF |
| 98 | GBM | primary | GBM | IV | f | 55 | n | y | y | y | N/A | CyTOF |
| 99 | GBM | primary | GBM | IV | m | 67 | n | y | y | n | N/A | IF/IHC |
| 100 | GBM | primary | GBM | IV | m | 59 | n | y | y | n | N/A | IF/IHC |
| 101 | GBM | primary | GBM | IV | m | 67 | n | y | y | n | N/A | IF/IHC |
| 102 | GBM | primary | GBM | IV | m | 68 | n | y | y | n | N/A | IF/IHC |
| 103 | GBM | primary | GBM | IV | f | 62 | n | y | y | n | N/A | IF/IHC |
| 104 | GBM | primary | GBM | IV | m | 50 | n | n | n | n | N/A | IF/IHC |
| 105 | GBM | primary | GBM | IV | m | 77 | n | y | y | n | N/A | IHC |

ICT: Immune Checkpoint Therapy,
CT: Chemotherapy,
RT: Radiation Therapy,
TT: Targeted Therapy,
HT: Hormonal Therapy

Supplementary Table 2: Table summarizing the statistical tests

| | | | | | | | | Multiple | |
|---|---|---|---|---|---|---|---|---|---|
| | | Number of | Type of | Statistical | Degrees | | Test | comparison | |
| Figure | Comparison | replicates | replicate | Test | of Freedom | P value | statistic | correction | Q value |
| 1a | GBM vs NSCLC | 8 vs 15 | patients | MW | n/a | 0.0025 | n/a | BH | 0.0033 |
| 1a | GBM vs RCC | 8 vs 11 | patients | MW | n/a | 0.0003 | n/a | BH | 0.0012 |
| 1a | GBM vs CRC | 8 vs 11 | patients | MW | n/a | 0.0018 | n/a | BH | 0.0033 |
| 1a | GBM vs PCa | 8 vs 21 | patients | MW | n/a | 0.2574 | n/a | BH | 0.2574 |
| 1d - L3 | GBM vs NSCLC vs RCC | 7 vs 11 vs 11 | patients | KW | n/a | 0.1172 | 7.379 | BH | 0.2051 |
| | vs CRC vs PCa | vs 11 vs 5 | | | | | | | |
| 1d - L12 | GBM vs NSCLC vs RCC | 7 vs 11 vs 11 | patients | KW | n/a | 0.0008 | 19.04 | BH | 0.0056 |
| | vs CRC vs PCa | vs 11 vs 5 | | | | | | | |
| 1d - L6 | GBM vs NSCLC vs RCC | 7 vs 11 vs 11 | patients | KW | n/a | 0.1347 | 7.023 | BH | 0.2095 |
| | vs CRC vs PCa | vs 11 vs 5 | | | | | | | |
| 1d - L14 | GBM vs NSCLC vs RCC | 7 vs 11 vs 11 | patients | KW | n/a | 0.0121 | 12.84 | BH | 0.0420 |
| | vs CRC vs PCa | vs 11 vs 5 | | | | | | | |
| 1d - L12 | CRC vs PCa | 11 vs 5 | patients | MW | n/a | 0.0197 | n/a | BH | 0.0262 |
| 1d - L12 | RCC vs CRC | 11 vs 11 | patients | MW | n/a | 0.0081 | n/a | BH | 0.0162 |
| 1d - L12 | NSCLC vs CRC | 11 vs 11 | patients | MW | n/a | 0.4772 | n/a | BH | 0.4772 |
| 1d - L12 | GBM vs CRC | 7 vs 11 | patients | MW | n/a | 0.0006 | n/a | BH | 0.0024 |
| 1d - L14 | PCa vs CRC | 5 vs 11 | patients | MW | n/a | 0.1162 | n/a | BH | 0.1162 |
| 1d - L14 | PCa vs RCC | 5 vs 11 | patients | MW | n/a | 0.0179 | n/a | BH | 0.0238 |
| 1d - L14 | PCa vs NSCLC | 5 vs 11 | patients | MW | n/a | 0.0179 | n/a | BH | 0.0238 |
| 1d - L14 | PCa vs GBM | 5 vs 7 | patients | MW | n/a | 0.0179 | n/a | BH | 0.0238 |
| 1F-T1 | I vs II | 11 vs 8 | patients | MW | n/a | 0.003 | n/a | BH | 0.0008 |
| 1F-T1 | II vs III | 8 vs 9 | patients | MW | n/a | <0.0001 | n/a | BH | 0.0008 |
| 1F-T4 | I vs II | 11 vs 8 | patients | MW | n/a | 0.0522 | n/a | BH | 0.0574 |
| 1F-T4 | II vs III | 8 vs 9 | patients | MW | n/a | 0.0023 | n/a | BH | 0.0074 |
| 1F-T17 | I vs II | 11 vs 8 | patients | MW | n/a | 0.0502 | n/a | BH | 0.1200 |
| 1F-T17 | II vs III | 8 vs 9 | patients | MW | n/a | 0.0498 | n/a | BH | 0.1200 |
| 1F-T1 | I vs II vs III | 11 vs 8 vs 9 | patients | KW | n/a | <0.0001 | 19.857 | BH | 0.0007 |
| 1F-T2 | I vs II vs III | 11 vs 8 vs 9 | patients | KW | n/a | 0.0082 | 9.5963 | BH | 0.0412 |
| 1F-T3 | I vs II vs III | 11 vs 8 vs 9 | patients | KW | n/a | 0.1535 | 3.7477 | BH | 0.2389 |
| 1F-T4 | I vs II vs III | 11 vs 8 vs 9 | patients | KW | n/a | 0.0015 | 12.893 | BH | 0.0118 |
| 1F-T17 | I vs II vs III | 11 vs 8 vs 9 | patients | KW | n/a | 0.07213 | 5.2586 | BH | 0.1801 |
| 2A - L8 | GBM vs NSCLC vs RCC | 7 vs 11 vs 11 | patients | KW | n/a | 0.0150 | 12.34 | BH | 0.042 |
| | vs CRC vs PCa | vs 11 vs 5 | | | | | | | |
| 2A - L1 | GBM vs NSCLC vs RCC | 7 vs 11 vs 11 | patients | KW | n/a | 0.2488 | 5.398 | BH | 0.042 |
| | vs CRC vs PCa | vs 11 vs 5 | | | | | | | |
| 2A - L17 | GBM vs NSCLC vs RCC | 7 vs 11 vs 11 | patients | KW | n/a | 0.0001 | 22.87 | BH | 0.0014 |
| | vs CRC vs PCa | vs 11 vs 5 | | | | | | | |
| 2A - L5 | GBM vs NSCLC vs RCC | 7 vs 11 vs 11 | patients | KW | n/a | 0.0052 | 14.76 | BH | 0.0243 |
| | vs CRC vs PCa | vs 11 vs 5 | | | | | | | |
| 2A - L8 | GBM vs NSCLC | 7 vs 11 | patients | MW | n/a | 0.0025 | n/a | BH | 0.01 |
| 2A - L8 | GBM vs RCC | 7 vs 11 | patients | MW | n/a | 0.0284 | n/a | BH | 0.0378 |
| 2A - L8 | GBM vs CRC | 7 vs 11 | patients | MW | n/a | 0.0163 | n/a | BH | 0.0326 |
| 2A - L8 | GBM vs PCa | 7 vs 5 | patients | MW | n/a | 0.0833 | n/a | BH | 0.0833 |
| 2A - L17 | CRC vs PCa | 11 vs 5 | patients | MW | n/a | 0.0211 | n/a | BH | 0.0211 |
| 2A - L17 | RCC vs CRC | 11 vs 11 | patients | MW | n/a | 0.0010 | n/a | BH | 0.0040 |
| 2A - L17 | NSCLC vs CRC | 11 vs 11 | patients | MW | n/a | 0.0027 | n/a | BH | 0.0054 |
| 2A - L17 | GBM vs CRC | 7 vs 11 | patients | MW | n/a | 0.0069 | n/a | BH | 0.0092 |
| 2A - L5 | CRC vs PCa | 11 vs 5 | patients | MW | n/a | 0.0936 | n/a | BH | 0.1248 |
| 2A - L5 | RCC vs CRC | 11 vs 11 | patients | MW | n/a | 0.0013 | n/a | BH | 0.0052 |
| 2A - L5 | NSCLC vs CRC | 11 vs 11 | patients | MW | n/a | 0.4863 | n/a | BH | 0.4863 |
| 2A - L5 | GBM vs CRC | 7 vs 11 | patients | MW | n/a | 0.004 | n/a | BH | 0.0080 |
| 3B | CD73hi vs CD7310 | 263 vs 263 | Patient | Logrank | 1 | 0.0131 | 6.151 | n/a | n/a |
| 4B | Wt vs KO untreated | 10 vs 9 | mice | Logrank | 1 | 0.0113 | 6.413 | n/a | n/a |
| 4B | Wt vs KO anti-PD-1 | 10 vs 11 | mice | Logrank | 1 | 0.1852 | 1.756 | n/a | n/a |
| 4B | Wt vs KO anti-PD-1 anti- | 13 vs 10 | mice | Logrank | 1 | 0.0348 | 4.456 | n/a | n/a |
| | CTLA-4 | | | | | | | | |
| 4B | KO untreated vs KO anti- | 9 vs 10 | mice | Logrank | 1 | <0.0001 | 16.52 | n/a | n/a |
| | PD-1 anti-CTLA-4 | | | | | | | | |
| 4D Upper | Wt vs KO untreated | 5 vs 5 | mice | t-test | 8 | 0.0417 | 2.422 | n/a | n/a |
| panel | | | | | | | | | |
| 4D Upper | Wt vs KO anti-PD-1 | 6 vs 5 | mice | t-test | 9 | 0.0521 | 2.237 | n/a | n/a |
| panel | | | | | | | | | |
| 4D Upper | Wt vs KO anti-PD-1 anti- | 5 vs 5 | mice | t-test | 7 | 0.0356 | 2.596 | n/a | n/a |
| panel | CTLA-4 | | | | | | | | |
| 4D Lower | Wt vs KO untreated | 5 vs 5 | mice | t-test | 8 | 0.0453 | 2.370 | n/a | n/a |
| panel | | | | | | | | | |
| 4D Lower | Wt vs KO anti-PD-1 | 6 vs 5 | mice | t-test | 9 | 0.0425 | 2.361 | n/a | n/a |
| panel | | | | | | | | | |
| 4D Lower | Wt vs KO anti-PD-1 anti- | 5 vs 5 | mice | t-test | 7 | 0.0292 | 2.733 | n/a | n/a |
| panel | CTLA-4 | | | | | | | | |
| Ext. Data | R T0 VS R T2 | 7 vs 6 | patients | MW | n/a | 0.0047 | n/a | n/a | 0.0140 |
| 3B - P33 | | | | | | | | | |
| Ext. Data | R T0 VS R T4 | 7 vs 4 | patients | MW | n/a | 0.0242 | n/a | n/a | 0.0360 |
| 3B - P33 | | | | | | | | | |
| Ext. Data | R T0 Vs R T2 | 7 vs 6 | patients | MW | n/a | 0.0023 | n/a | n/a | 0.0070 |
| 3B - P24 | | | | | | | | | |
| Ext. Data | R T0 VS R T4 | 7 vs 4 | patients | MW | n/a | 0.0242 | n/a | n/a | 0.0360 |
| 3B - P24 | | | | | | | | | |
| Ext. Data | R T0 vs R T2 vs R T4 | 7 vs 6 vs 4 | patients | KW | n/a | 0.0031 | 9.341 | n/a | 0.0473 |
| 3B - P33 | | | | | | | | | |
| Ext. Data | R T0 vs R T2 vs R T4 | 7 vs 6 vs 4 | patients | KW | n/a | 0.0013 | 10.30 | n/a | 0.0429 |
| 3B - P24 | | | | | | | | | |
| Ext. Data 5C | GBM vs PCa | 7 vs 5 | patients | MW | n/a | 0.0025 | n/a | BH | 0.0031 |
| Ext. Data 5C | GBM vs mCRC | 7 vs 3 | patients | MW | n/a | 0.0061 | n/a | BH | 0.0061 |
| Ext. Data 5C | GBM vs pCRC | 7 vs 7 | patients | MW | n/a | 0.0006 | n/a | BH | 0.0010 |
| Ext. Data 5C | GBM vs RCC | 7 vs 11 | patients | MW | n/a | 0.0004 | n/a | BH | 0.0010 |
| Ext. Data 5C | GBM vs NSCLC | 7 vs 11 | patients | MW | n/a | <0.0001 | n/a | BH | 0.0005 |
| Ext. Data 5F | CD68+ vs CD8+ | 7 vs 7 | patients | MW | n/a | 0.0156 | n/a | BH | 0.0156 |
| Ext. Data 5F | CD68+ vs CD3+ | 7 vs 7 | patients | MW | n/a | 0.0156 | n/a | BH | 0.0156 |
| Ext. Data 8A | Wt vs KO | 7 vs 7 | Mice | MW | n/a | 0.01 | n/a | n/a | n/a |
| Ext. Data 8B | Wt vs KO | 7 vs 7 | Mice | Logrank | n/a | 0.0113 | 6.413 | n/a | n/a |
| Ext. Data | Wt vs ko | 5 vs 5 | Mice | MW | n/a | 0.0079 | n/a | n/a | 0.1580 |
| 8D-Gmm20 | | | | | | | | | |
| 8D-Gmm13 | Wt vs ko | 5 vs 5 | Mice | MW | n/a | 0.0159 | n/a | n/a | 0.1600 |

BH: Benjamini-Hochberg, CRC: colorectal adenocarcinoma, GBM: glioblastoma multiforme, KO: CD73 knockout, KW: Kruskal-Wallis test, MW: Mann-Whitney test, WSR: Wilcoxon signed rank test, n/a: not applicable; NSCLC: non small-cell lung cancer, PCa: prostate adenocarcinoma, RCC: renal cell carcinoma, wt: CD73 wild type.

Supplementary Table 3: EMD values
and associated z-scores of genes

| Gene | EMD | Z-score |
|---|---|---|
| SNX10 | 10.08489 | 4.252534 |
| TSPAN4 | 9.168137 | 3.828317 |
| BLVRA | 9.145905 | 3.818029 |
| CLEC11A | 8.864722 | 3.687916 |
| ACTN1 | 8.778338 | 3.647943 |
| SNAI1 | 8.776704 | 3.647187 |
| SPNS1 | 8.703557 | 3.613339 |
| EMILIN2 | 8.599669 | 3.565267 |
| KCNN4 | 8.550683 | 3.542599 |
| CPNE8 | 8.511295 | 3.524372 |
| ANXA2 | 8.502675 | 3.520384 |
| MMGT1 | 8.367893 | 3.458015 |
| ARL8B | 8.345225 | 3.447526 |
| GRN | 8.317021 | 3.434475 |
| CATSPER1 | 8.21103 | 3.385429 |
| HK3 | 8.134035 | 3.349801 |
| CLEC5A | 8.130381 | 3.34811 |
| DAPK1 | 8.130111 | 3.347985 |
| LATS2 | 8.03568 | 3.304288 |
| S100A10 | 8.022549 | 3.298212 |
| ASAP1 | 7.984883 | 3.280783 |
| VIM | 7.952731 | 3.265905 |
| GABBR1 | 7.950721 | 3.264975 |
| MARVELD1 | 7.901878 | 3.242374 |
| LGALS1 | 7.867662 | 3.22654 |
| FES | 7.733672 | 3.164538 |
| IMPDH1 | 7.716943 | 3.156797 |
| TRPS1 | 7.694777 | 3.14654 |
| NCF2 | 7.677418 | 3.138508 |
| AP1S2 | 7.636328 | 3.119494 |
| ATP6V0D1 | 7.6355 | 3.119111 |
| DENND6B | 7.629788 | 3.116468 |
| SHB | 7.581894 | 3.094306 |
| GRINA | 7.541816 | 3.07576 |
| TIMP4 | 7.504752 | 3.058609 |
| SPG21 | 7.447277 | 3.032014 |
| H2AFY | 7.429388 | 3.023736 |
| S100A6 | 7.415963 | 3.017523 |
| FUOM | 7.411658 | 3.015531 |
| MXD1 | 7.410833 | 3.01515 |
| FNDC3B | 7.399791 | 3.01004 |
| SPINT1 | 7.393085 | 3.006937 |
| SIGLEC7 | 7.392237 | 3.006545 |
| COMT | 7.379043 | 3.000439 |

Supplementary Table 4: Key Resource Table

| Reagent or Resource | Source | Identifier | Dilution |
|---|---|---|---|
| Antibodies for human CyTOF | | | |
| Anti-B7-H3 (clone 185504) | R&D | MAB1027-500 | 1:200 |
| Anti-B7-H4 (clone 9M1-3) | eBioscience | Cat# 16-5949-82 | 1:100 |
| Anti-BTLA (clone J168-540) | Fluidigm | Cat# 3163009B | 1:100 |
| Anti-CD3 (clone UCTH1) | Biolegend | Cat# 300443 | 1:100 |
| Anti-CD4 (clone RPA-T4) | Fluidigm | Cat# 3145001B | 1:100 |
| Anti-CD8a (clone RPA-T8) | Fluidigm | Cat# 3146001B | 1:100 |
| Anti-CD27 (clone LI28) | Fluidigm | Cat# 3155001B | 1:100 |
| Anti-CD28 (clone CD28.2) | Fluidigm | Cat# 3160003B | 1:100 |
| Anti-CD45 (clone HI30) | Fluidigm | Cat# 3089003B | 1:400 |
| Anti-CD56 (clone NCAM16.2) | Fluidigm | Cat# 3176008B | 1:100 |
| Anti-CD68 (clone Y1/82A) | Biolegend | Cat# 333802 | 1:200 |
| Anti-CD68 (clone PG-M1) | Dako | Cat# M0876 | 1:200 |
| Anti-CD70 (clone BU69) | Ancell | Cat# 222-820 | 1:100 |
| Anti-CD73 (clone AD2) | Biolegend | Cat# 344002 | 1:100 |
| Anti-CD73 (clone 1D7) | Abcam | Cat# ab91086 | 1:100 |
| Anti-CD80 (clone L307.4) | BD Biosciences | Cat# 557223 | 1:100 |
| Anti-CD86 (clone IT2.2) | Fluidigm | Cat# 3156008B | 1:100 |
| Anti-CD137 (clone 4B4-1) | Biolegend | Cat# 309802 | 1:100 |
| Anti-CD137L (clone C65-485) | BD Biosciences | Cat# 559445 | 1:100 |
| Anti-CD163 (clone 10D6) | Leica Biosystems | Cat# NCL-L-CD163 | 1:100 |
| Anti-CD326 (clone 9C4) | Fluidigm | Cat# 3141006B | 1:100 |
| Anti-CTLA-4 (clone 14D3) | Fluidigm | Cat# 3170005B | 1:100 |
| Anti-FoxP3 (clone PCH101) | Fluidigm | Cat# 3162011A | 1:100 |
| Anti-Galectin 9 (clone 9M1-3) | Biolegend | Cat# 348902 | 1:100 |
| Anti-GITR (clone 621) | Biolegend | Cat# 311606 | 1:100 |
| Anti-HLA-DR (clone L243) | Fluidigm | custom | 1:400 |
| Anti-HLA-DR (clone L243) | Fluidigm | Cat# 3174001B | 1:400 |
| Anti-HVEM (clone ANC3B7) | Ancell | Cat# 270-820 | 1:100 |
| Anti-ICOS (clone ISA-3) | eBioscience | Cat# 14-9948-82 | 1:100 |
| Anti-ICOSL (clone 2D3) | BD Biosciences | Cat# 552501 | 1:100 |
| Anti-Ki67 (clone B56) | Biolegend | Cat# 350523 | 1:100 |
| Anti-LAG3 (clone 874501) | R&D | Cat# MAB23193SP | 1:50 |
| Anti-OX40 (clone ACT35) | Biolegend | Cat# 350015 | 1:100 |
| Anti-OX40L (clone IK-1) | BD Biosciences | custom | 1:100 |
| Anti-PD-1 (clone EH12.2H7) | Biolegend | Cat# 329941 | 1:100 |
| Anti-PD-L1 (clone MIH1) | eBioscience | Cat# 14-5983-82 | 1:100 |
| Anti-PD-L2 (clone 24F.10C12) | Fluidigm | Cat# 3172014B | 1:100 |
| Anti-TIGIT (clone MBSA43) | eBioscience | Cat# 16-9500-85 | 1:100 |
| Anti-TIM-3 (clone F38-2E2) | Fluidigm | Cat# 3153008B | 1:200 |
| Anti-VISTA (clone 730804) | R&D | Cat# MAB71261 | 1:100 |
| Surgical samples | | | |
| Tumor surgical samples | MDACC | See Table S2 | |
| Chemicals | | | |
| Antibody Stabilizer | Candor | Cat# 131 050 | |
| | Biosciences | | |
| Cisplatin | Fluidigm | Cat# 201064 | |
| Cy3 | PerkinElmer | Cat# NEL744B001KT | |
| Cy5 | PerkinElmer | Cat# NEL745B001KT | |
| Cy5.5 | PerkinElmer | Cat# NEL766B001KT | |
| DAPI | PerkinElmer | Cat# FP1490 | |
| EQ 4-element beads | Fluidigm | Cat# 201078 | |
| Ir DNA-Intercalator | Fluidigm | Cat# 201192A | |
| VECTASHIELD HardSet Antifade | Vector Laboratories | Cat# H-1400 | |
| Mounting Medium | | | |
| Commercial Assays | | | |
| X8 Antibody Labeling Kit | Fluidigm | N/A (metal specific) | |
| Deposited Data | | | |
| Mass cytometry data | FlowRepository | FR-FCM-Z2B3 | |
| Single Cell RNA sequencing | Sequenced Read | PRJNA588461 | |
| | Archive (SRA) | | |

| Antibodies for PBMC CyTOF | Source | Identifier | Dilution |
|---|---|---|---|
| Anti-B7-H3 (clone 185504) | R&D | MAB1027-500 | 1:200 |
| Anti-B7-H4 (clone 9M1-3) | eBioscience | Cat# 16-5949-82 | 1:100 |
| Anti-BTLA (clone J168-540) | Fluidigm | Cat# 3163009B | 1:100 |
| Anti-CD3 (clone UCTH1) | Biolegend | Cat# 300443 | 1:100 |
| Anti-CD4 (clone RPA-T4) | Fluidigm | Cat# 3145001B | 1:100 |
| Anti-CD8a (clone RPA-T8) | Fluidigm | Cat# 3146001B | 1:100 |
| Anti-CD14 (clone M5E2) | Biolegend | Cat# 301802 | 1:100 |
| Anti-CD19 (clone HIB19) | Biolegend | Cat# 302202 | 1:167 |
| Anti-CD27 (clone LI28) | Fluidigm | Cat# 3155001B | 1:100 |
| Anti-CD28 (clone CD28.2) | Fluidigm | Cat# 3160003B | 1:100 |
| Anti-CD45 (clone HI30) | Fluidigm | Cat# 3089003B | 1:400 |
| Anti-CD45RA (clone HI100) | Fluidigm | Cat# 3155011B | 1:200 |
| Anti-CD45RO (clone UCHL1) | Biolegend | Cat# 304202 | 1:200 |
| Anti-CD56 (clone NCAM16.2) | Fluidigm | Cat# 3176008B | 1:100 |
| Anti-CD68 (clone Y1/82A) | Biolegend | Cat# 333802 | 1:200 |
| Anti-CD73 (clone AD2) | Biolegend | Cat# 344002 | 1:100 |
| Anti-CD86 (clone IT2.2) | Fluidigm | Cat# 3156008B | 1:100 |
| Anti-CD137 (clone 4B4-1) | Biolegend | Cat# 309802 | 1:100 |
| Anti-CCR7 (clone G043H7) | Biolegend | Cat# 353237 | 1:125 |
| Anti-CTLA-4 (clone 14D3) | Fluidigm | Cat# 3170005B | 1:100 |
| Anti-Eomes (clone WD1928) | eBioscience | Cat# 12-4877-42 | 1:167 |
| Anti-FoxP3 (clone PCH101) | Fluidigm | Cat# 3162011A | 1:100 |
| Anti-GITR (clone 621) | Biolegend | Cat# 311606 | 1:100 |
| Anti-HLA-DR (clone L243) | Fluidigm | custom | 1:400 |
| Anti-HVEM (clone ANC3B7) | Ancell | Cat# 270-820 | 1:100 |
| Anti-ICOS (clone ISA-3) | eBioscience | Cat# 14-9948-82 | 1:100 |
| Anti-ICOSL (clone 2D3) | BD Biosciences | Cat# 552501 | 1:100 |
| Anti-Ki67 (clone B56) | Biolegend | Cat# 350523 | 1:100 |
| Anti-LAG3 (clone 874501) | R&D | Cat# MAB23193SP | 1:50 |
| Anti-OX40 (clone ACT35) | Biolegend | Cat# 350015 | 1:100 |
| Anti-PD-1 (clone EH12.2H7) | Biolegend | Cat# 329941 | 1:100 |
| Anti-PD-L1 (clone MIH1) | eBioscience | Cat# 14-5983-82 | 1:100 |
| Anti-PD-L2 (clone 24F.10C12) | Fluidigm | Cat# 3172014B | 1:100 |
| Anti-Tbet (clone 4B10) | Biolegend | Cat# 644802 | 1:200 |
| Anti-TIGIT (clone MBSA43) | eBioscience | Cat# 16-9500-85 | 1:100 |
| Anti-TIM-3 (clone F38-2E2) | Fluidigm | Cat# 3153008B | 1:200 |
| Anti-VISTA (clone 730804) | R&D | Cat# MAB71261 | 1:100 |

| Antibodies for murine CyTOF | Source | Identifier | Dilutions |
|---|---|---|---|
| Anti-CD45 (clone 30-F11) | Fluidigm | Cat# 3089005B | 1:200 |
| Anti-I-A/I-E (clone M5/114.15.2) | Biolegend | Cat# 107637 | 1:800 |
| Anti-CD4 (clone RMP4-5) | Biolegend | Cat# 100506 | 1:200 |
| Anti-CD73 (clone TY/11.8) | Biolegend | Cat# 127202 | 1:400 |
| Anti-Ly6G (clone 1A8) | Biolegend | Cat# 127620 | 1:200 |
| Anti-Tbet (clone 4B10) | Biolegend | Cat# 644805 | 1:150 |
| Anti-VISTA (clone MIH63) | Biolegend | Cat# 150202 | 1:200 |
| Anti-CD204 (clone 7G5C33) | Biolegend | Cat# 154702 | 1:100 |
| Anti-PD-L2 (clone Ty25) | Biolegend | Cat# 107202 | 1:100 |
| Anti-CD103 (clone 2E7) | Biolegend | Cat# 121402 | 1:200 |
| Anti-CCR2 (clone MAB55381) | Biolegend | Cat# 475301 | 1:200 |
| Anti-Eomes (clone Dan11mag) | eBioscience | Cat# 14-4875-82 | 1:100 |
| Anti-CD80 (clone 16-10A1) | Biolegend | Cat# 104710 | 1:800 |
| Anti-CD163 (clone EPR19518) | abcam | Cat# ab182422 | 1:50 |
| Anti-GATA3 (clone TWAJ) | eBioscience | Cat# 14-9966-82 | 1:100 |
| Anti-ICOS (clone 7E.17G9) | eBioscience | Cat# 14-9942-85 | 1:100 |
| Anti-F4/80 (clone BM8) | Biolegend | Cat# 123143 | 1:100 |
| Anti-CD86 (clone GL-1) | Biolegend | Cat# 105002 | 1:100 |
| Anti-GranzymeB (clone QA16A02) | Biolegend | Cat# 372202 | 1:100 |
| Anti-CD115 (clone AFS98) | Fluidigm | Cat# 3144012B | 1:50 |
| Anti-FoxP3 (clone FJK-16s) | Fluidigm | Cat# 3158003A | 1:100 |
| Anti- CD8a (clone 53-6.7) | Fluidigm | Cat# 3146003B | 1:200 |
| Anti- CD19 (clone 6D5) | Fluidigm | Cat# 3149002B | 1:200 |
| Anti-Ly6C (clone HK1.4) | Fluidigm | Cat# 3150010B | 1:400 |
| Anti-CD25 (clone 3C7) | Fluidigm | Cat# 3151007B | 1:50 |
| Anti-CD3e (clone 145-2C11) | Fluidigm | Cat# 3152004B | 1:100 |
| Anti-CTLA-4 (clone UC10-4B9) | Fluidigm | Cat# 3154008B | 1:100 |
| Anti-LAG3 (clone C9B7W) | Fluidigm | Cat# 3174019B | 1:100 |
| Anti- iNOS (clone CXNFT) | Fluidigm | Cat# 3161011B | 1:100 |
| Anti-PD-1 (clone J43) | Fluidigm | Cat# 3159023B | 1:100 |
| Anti-CD206 (clone C068C2) | Fluidigm | Cat# 3169021B | 1:200 |
| Anti-NK1.1 (clone PK136) | Fluidigm | Cat# 3170002B | 1:100 |
| Anti-CD11b (clone M1/70 ) | Fluidigm | Cat# 3172012B | 1:1600 |
| Anti-Arginase1 (clone 8C9) | Santacruz | Cat# sc-47715 | 1:300 |
| Anti-CD11c (clone N418 ) | Fluidigm | Cat# 3153016B | 1:50 |
| Anti-PD-L1 (clone 10F.9G2) | Fluidigm | Cat# 3142003B | 1:200 |

Software

| Aperio ImageScope | Leica Biosystems | N/A |
|---|---|---|
| Bead Normalization | (Finck et al., 2013) | R package premessa (Parker Institute for Cancer |
| | | Immunotherapy) |
| Cytobank | (Kotecha et al., 2010) | Available on the world wide web at cytobank.org/ |
| FlowJo | Tree Star, Inc. | Available on the world wide web at flowjo.com/ |
| Helios software 6.5.358 | Fluidigm | Available on the world wide web at |
| | | fluidigm.com/software |
| MAGIC | (van Dijk et al., 2018) | |
| MATLAB R2016a | Mathworks | mathworks.com |
| Phenograph | (Levine et al., 2015) | Available on the world wide web at |
| | | github.com/jacoblevine/PhenoGraph |
| Prism 7.0 | GraphPad | N/A |
| R 3.3.2 | R Development Core | Available on the world wide web at r-project.org/ |
| | Team, 2015 | |
| SEQC | (Azizi et al., 2018) | Available on the world wide web at |
| | | github.com/ambrosejcarr/seqc) |
| t-SNE | (van der Maaten and | Available on the world wide web at |
| | Hinton, 2008) | github.com/jkrijthe/Rtsne |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the publications referred to throughout the specification, to the extent that they provide exemplary procedural or other details supplementary to those set forth herein, are specifically incorporated herein by reference.
1. Sharma, P. & Allison, J. P. Immune checkpoint targeting in cancer therapy: toward combination strategies with curative potential. Cell 161, 205-214 (2015).
2. Sharma, P. & Allison, J. P. The future of immune checkpoint therapy. Science 348, 56-61(2015).
3. Borghaei, H., et al. Nivolumab versus Docetaxel in Advanced Nonsquamous Non-Small-Cell Lung Cancer. The New England journal of medicine 373, 1627-1639 (2015).
4. Hodi, F. S., et al. Improved survival with ipilimumab in patients with metastatic melanoma. The New England journal of medicine 363, 711-723 (2010).
5. Kwon, E. D., et al. Ipilimumab versus placebo after radiotherapy in patients with metastatic castration-resistant prostate cancer that had progressed after docetaxel chemotherapy (CA184-043): a multicentre, randomised, double-blind, phase 3 trial. The Lancet. Oncology 15, 700-712 (2014).
6. Motzer, R. J., et al. Nivolumab versus Everolimus in Advanced Renal-Cell Carcinoma. The New England journal of medicine 373, 1803-1813 (2015).
7. Motzer, R. J., et al. Nivolumab plus Ipilimumab versus Sunitinib in Advanced Renal-Cell Carcinoma. The New England journal of medicine 378, 1277-1290 (2018).
8. Sharma, P., et al. Nivolumab in metastatic urothelial carcinoma after platinum therapy (CheckMate 275): a multicentre, single-arm, phase 2 trial. The Lancet. Oncology 18, 312-322 (2017).
9. Le, D. T., et al. Mismatch-repair deficiency predicts response of solid tumors to PD-1 blockade. Science (2017).
10. Chevrier, S., et al. An Immune Atlas of Clear Cell Renal Cell Carcinoma. Cell 169, 736-749 e718 (2017).
11. Chew, V., et al. Delineation of an immunosuppressive gradient in hepatocellular carcinoma using high-dimensional proteomic and transcriptomic analyses. Proceedings of the National Academy of Sciences of the United States of America 114, E5900-E5909 (2017).
12. Lavin, Y., et al. Innate Immune Landscape in Early Lung Adenocarcinoma by Paired Single-Cell Analyses. Cell 169, 750-765 e717 (2017).
13. Wei, S. C., et al. Distinct Cellular Mechanisms Underlie Anti-CTLA-4 and Anti-PD-1 Checkpoint Blockade. Cell 170, 1120-1133 e1117 (2017).
14. Gao, J., et al. Loss of IFN-gamma Pathway Genes in Tumor Cells as a Mechanism of Resistance to Anti-CTLA-4 Therapy. Cell 167, 397-404 e399 (2016).
15. Saito, T., et al. Two FOXP3(+)CD4(+) T cell subpopulations distinctly control the prognosis of colorectal cancers. Nature medicine 22, 679-684 (2016).
16. Antonioli, L., Pacher, P., Vizi, E. S. & Hasko, G. CD39 and CD73 in immunity and inflammation. Trends Mol Med 19, 355-367 (2013).
17. Azambuja, J. H., et al. CD73 Downregulation Decreases In Vitro and In Vivo Glioblastoma Growth. Mol Neurobiol 56, 3260-3279 (2019).
18. Azambuja, J. H., et al. Nasal Administration of Cationic Nanoemulsions as CD73-siRNA Delivery System for Glioblastoma Treatment: a New Therapeutical Approach. Mol Neurobiol (2019).
19. Takenaka, M. C., et al. Control of tumor-associated macrophages and T cells in glioblastoma via AHR and CD39. Nat Neurosci 22, 729-740 (2019).
20. Yan, A., et al. CD73 Promotes Glioblastoma Pathogenesis and Enhances Its Chemoresistance via A2B Adenosine Receptor Signaling. J Neurosci 39, 4387-4402 (2019).
21. Muller, S., et al. Single-cell profiling of human gliomas reveals macrophage ontogeny as a basis for regional differences in macrophage activation in the tumor microenvironment. Genome Biol 18, 234 (2017).
22. Chang, A. L., et al. CCL2 Produced by the Glioma Microenvironment Is Essential for the Recruitment of Regulatory T Cells and Myeloid-Derived Suppressor Cells. Cancer research 76, 5671-5682 (2016).
23. Chen, Z., et al. Cellular and Molecular Identity of Tumor-Associated Macrophages in Glioblastoma. Cancer research 77, 2266-2278 (2017).
24. Pham, K., Luo, D., Liu, C. & Harrison, J. K. CCL5, CCR1 and CCR5 in murine glioblastoma: immune cell infiltration and survival rates are not dependent on individual expression of either CCR1 or CCR5. J Neuroimmunol 246, 10-17 (2012).
25. Pyonteck, S. M., et al. CSF-1R inhibition alters macrophage polarization and blocks glioma progression. Nature medicine 19, 1264-1272 (2013).
26. Wang, Y., et al. Hypoxia and macrophages promote glioblastoma invasion by the CCL4-CCR5 axis. Oncol Rep 36, 3522-3528 (2016).
27. Schalper, K. A., et al. Neoadjuvant nivolumab modifies the tumor immune microenvironment in resectable glioblastoma. Nature medicine 25, 470-476 (2019).
28. Cloughesy, T. F., et al. Neoadjuvant anti-PD-1 immunotherapy promotes a survival benefit with intratumoral and systemic immune responses in recurrent glioblastoma. Nature medicine 25, 477-486 (2019).
29. Stagg, J., et al. CD73-deficient mice have increased antitumor immunity and are resistant to experimental metastasis. Cancer research 71, 2892-2900 (2011).
30. Andrews, L. P., Marciscano, A. E., Drake, C. G. & Vignali, D. A. LAG3 (CD223) as a cancer immunotherapy target. Immunol Rev 276, 80-96 (2017).
31. Larkin, J., et al. Combined Nivolumab and Ipilimumab or Monotherapy in Untreated Melanoma. The New England journal of medicine 373, 23-34 (2015).
32. Pfannenstiel, L. W., et al. Immune-Checkpoint Blockade Opposes CD8(+) T-cell Suppression in Human and Murine Cancer. Cancer immunology research 7, 510-525 (2019).
33. Papadopoulos, K. P., et al. First-in-Human Study of AMG 820, a Monoclonal Anti-Colony-Stimulating Factor 1 Receptor Antibody, in Patients with Advanced Solid Tumors. Clinical cancer research: an official journal of the American Association for Cancer Research 23, 5703-5710 (2017).
34. Cannarile, M. A., et al. Colony-stimulating factor 1 receptor (CSF1R) inhibitors in cancer therapy. J Immunother Cancer 5, 53 (2017).
35. Antonioli, L., et al. Switching off CD73: a way to boost the activity of conventional and targeted antineoplastic therapies. Drug Discov Today 22, 1686-1696 (2017).
36. Perrot, I., et al. Blocking Antibodies Targeting the CD39/CD73 Immunosuppressive Pathway Unleash Immune Responses in Combination Cancer Therapies. Cell reports 27, 2411-2425 e2419 (2019).
37. Wei, J., et al. MiR-138 exerts anti-glioma efficacy by targeting immune checkpoints. Neuro Oncol 18, 639-648 (2016).
38. Finck, R., et al. Normalization of mass cytometry data with bead standards. Cytometry A 83, 483-494 (2013).
39. Zunder, E. R., et al. Palladium-based mass tag cell barcoding with a doublet-filtering scheme and single-cell deconvolution algorithm. Nat Protoc 10, 316-333 (2015).
40. Amir el, A. D., et al. viSNE enables visualization of high dimensional single-cell data and reveals phenotypic heterogeneity of leukemia. Nature biotechnology 31, 545-552 (2013).
41. Levine, J. H., et al. Data-Driven Phenotypic Dissection of AML Reveals Progenitor-like Cells that Correlate with Prognosis. Cell 162, 184-197 (2015).
42. Van Gassen, S., et al. FlowSOM: Using self-organizing maps for visualization and interpretation of cytometry data. Cytometry A 87, 636-645 (2015).
43. Azizi, E., et al. Single-Cell Map of Diverse Immune Phenotypes in the Breast Tumor Microenvironment. Cell 174, 1293-1308 e1236 (2018).
44. van Dijk, D., et al. Recovering Gene Interactions from Single-Cell Data Using Data Diffusion. Cell 174, 716-729 e727 (2018).

What is claimed is:

1. A method of treating glioblastoma in a subject comprising administering to the subject immune checkpoint blockade (ICB) therapy after the subject has been determined to have low expression of CD73 in a biological sample from the subject.

2. The method of claim 1, wherein the expression is low as compared to a control.

3. The method of claim 1 or 2, wherein the biological sample comprises isolated immune cells.

4. The method of claim 3, wherein the biological sample comprises isolated macrophages.

5. The method of any one of claims 1-4, wherein the biological samples comprises a serum sample, a biopsy sample, or an isolated fraction of immune cells.

6. The method any one of claims 3-5, wherein the expression of CD73 is determined to be low in immune cells.

7. The method of any one of claims 1-6, wherein the ICB therapy comprises a monotherapy or a combination ICB therapy.

8. The method of any one of claims 1-7, wherein the ICB therapy comprises an inhibitor of PD-1, PDL1, PDL2, CTLA-4, B7-1, and/or B7-2.

9. The method of any one of claims 1-8, wherein the ICB therapy comprises an anti-PD-1 monoclonal antibody and/or an anti-CTLA-4 monoclonal antibody.

10. The method of claim 9, wherein the ICB therapy comprises one or more of nivolumab, pembrolizumab, pidilizumab, ipilimumab or tremelimumab.

11. The method of any one of claims 1-8, wherein the method further comprises administering at least one additional anticancer treatment.

12. The method of claim 11, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

13. The method of any one of claims 2-12, wherein the control comprises a cut-off value or a normalized value.

14. The method of any one of claims 1-13, wherein the low expression level comprises a normalized level of expression that is determined to be decreased as compared to a control.

15. The method of any one of claims 1-14, wherein the CD73 expression was detected by an immunoassay.

16. A method of treating glioblastoma in a subject comprising administering to the subject an agent selected from a CD73 inhibitor, a CD39 inhibitor, or an A2AR antagonist after the subject has been determined to have high expression of CD73 in a biological sample from the subject.

17. The method of claim 16, wherein the expression is determined to be high as compared to a control.

18. The method of claim 16 or 17, wherein the method further comprises administration of ICB therapy to the subject.

19. The method of claim 18, wherein the CD73 inhibitor, CD39 inhibitor, or A2AR antagonist is administered prior to the ICB therapy.

20. The method of claim 18, wherein the ICB therapy and CD73 inhibitor, CD39 inhibitor, or A2AR antagonist are administered simultaneously.

21. The method of any one of claims 16-20, wherein the CD73 or CD39 inhibitor comprises an anti-CD73 or an anti-CD39 antibody, respectively.

22. The method of claim 21, wherein the antibody comprises a blocking antibody and/or induces antibody-dependent cellular cytotoxicity.

23. The method of any one of claims 16-22, wherein the A2AR antagonist comprises ATL-444, Istradefylline (KW-6002), MSX-3, Preladenant (SCH-420,814), SCH-58261, SCH-412,348, SCH-442,416, ST-1535, Caffeine, VER-6623, VER-6947, VER-7835, Vipadenant (BIIB-014), ZM-241,385, or combinations thereof.

24. The method of any one of claims 16-23, wherein the biological sample comprises isolated immune cells.

25. The method of claim 24, wherein the biological sample comprises isolated macrophages.

26. The method of any one of claims 16-25, wherein the biological samples comprises a serum sample, a biopsy sample, or an isolated fraction of immune cells.

27. The method of any one of claims 16-26, wherein the expression of CD73 is determined to be high in immune cells.

28. The method of any one of claims 24-27, wherein the ICB therapy comprises a monotherapy or a combination ICB therapy.

29. The method of any one of claims 18-28, wherein the ICB therapy comprises an inhibitor of PD-1, PDL1, PDL2, CTLA-4, B7-1, and/or B7-2.

30. The method of any one of claims 18-29, wherein the ICB therapy comprises an anti-PD-1 monoclonal antibody and/or an anti-CTLA-4 monoclonal antibody.

31. The method of claim 30, wherein the ICB therapy comprises one or more of nivolumab, pembrolizumab, pidilizumab, ipilimumab or tremelimumab.

32. The method of any one of claims 16-29, wherein the method further comprises administering at least one additional anticancer treatment.

33. The method of claim 32, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

34. The method of any one of claims 17-33, wherein the control comprises a cut-off value or a normalized value.

35. The method of any one of claims 16-34, wherein the high expression level comprises level comprises a normalized level of expression that is determined to be high as compared to a control.

36. The method of any one of claims 16-35, wherein the CD73 expression was detected by an immunoassay.

37. A method for predicting a response to ICB therapy in a subject having glioblastoma, the method comprising:
(a) determining the expression level of CD73 in a sample from the subject;
(b) comparing the expression level of CD73 in a sample from the subject to a control; and
(c) predicting that the subject will respond to the ICB therapy after (i) a decreased expression level of CD73 is detected in a biological sample from the subject as compared to a control, wherein the control represents an expression level of CD73 in a biological sample from a subject that has been determined to not respond to ICB therapy; or (ii) a decreased or a non-significantly different expression level of CD73 is detected in a biological sample from the subject as compared to a control, wherein the control represents an expression level of CD73 in a biological sample from a subject that has been determined to respond to ICB therapy; or
(d) predicting that the subject will not respond to the ICB therapy after (i) an increased expression level of CD73 is detected in a biological sample from the subject as compared to a control, wherein the control represents an expression level of CD73 in a biological sample from a subject that has been determined to respond to ICB therapy; or (ii) an increase or a non-significantly different expression level of CD73 is detected in a biological sample from the subject as compared to a control, wherein the control represents an expression level of CD73 in a biological sample from a subject that has been determined to not respond to ICB therapy.

38. The method of claim 37, wherein the method further comprises treating the subject predicted to respond to ICB therapy with ICB therapy.

39. The method of claim 37 or 38, wherein the biological sample comprises isolated immune cells.

40. The method of claim 39, wherein the biological sample comprises isolated macrophages.

41. The method of any one of claims 37-40, wherein the biological samples comprises a serum sample, a biopsy sample, or an isolated fraction of immune cells.

42. The method of any one of claims 37-41, wherein the ICB therapy comprises a monotherapy or a combination ICB therapy.

43. The method of any one of claims 37-42, wherein the ICB therapy comprises an inhibitor of PD-1, PDL1, PDL2, CTLA-4, B7-1, and/or B7-2.

44. The method of any one of claims 37-43, wherein the ICB therapy comprises an anti-PD-1 monoclonal antibody and/or an anti-CTLA-4 monoclonal antibody.

45. The method of claim 44, wherein the ICB therapy comprises one or more of nivolumab, pembrolizumab, pidilizumab, ipilimumab or tremelimumab.

46. The method of any one of claims 37-45, wherein the method further comprises administering ICB therapy to a subject predicted to respond to ICB therapy.

47. The method of any one of claims 37-45, wherein the method further comprises administering a CD73 inhibitor, a CD39 inhibitor, or an A2AR antagonist to a subject predicted to not respond to ICB therapy.

48. The method of claim 47, wherein the method further comprises administration of ICB therapy to the subject.

49. The method of claim 48, wherein the CD73 inhibitor, CD39 inhibitor, or A2AR antagonist is administered prior to the ICB therapy.

50. The method of claim 48, wherein the ICB therapy and CD73 inhibitor, CD39 inhibitor, or A2AR antagonist are administered simultaneously.

51. The method of any one of claims 47-50, wherein the CD73 or CD39 inhibitor comprises an anti-CD73 or an anti-CD39 antibody, respectively.

52. The method of claim 51, wherein the antibody comprises a blocking antibody and/or induces antibody-dependent cellular cytotoxicity.

53. The method of any one of claims 47-52, wherein the A2AR antagonist comprises ATL-444, Istradefylline (KW-6002), MSX-3, Preladenant (SCH-420,814), SCH-58261, SCH-412,348, SCH-442,416, ST-1535, Caffeine, VER-6623, VER-6947, VER-7835, Vipadenant (BIIB-014), ZM-241,385, or combinations thereof.

54. The method of any one of claims 37-53, wherein the method further comprises administering at least one additional anticancer treatment.

55. The method of claim 54, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

56. The method of any one of claims 37-55, wherein the control comprises a cut-off value or a normalized value.

57. The method of any one of claims 37-56, wherein the expression level comprises a normalized level of expression.

58. The method of any one of claims 37-57, wherein the CD73 expression was detected by an immunoassay.

59. A method comprising detecting CD73 in a biological sample from a subject with glioblastoma.

60. The method of claim 59, wherein the biological sample comprises isolated immune cells.

61. The method of claim 60, wherein the biological sample comprises isolated macrophages.

62. The method of any one of claims 59-61, wherein the biological samples comprises a serum sample, a biopsy sample, or an isolated fraction of immune cells.

63. The method of any one of claims 59-62, wherein the control comprises a cut-off value or a normalized value.

64. The method of any one of claims 59-63, wherein the expression level comprises a normalized level of expression.

65. The method of any one of claims 59-64, wherein the CD73 expression was detected by an immunoassay.

66. The method of any one of claims 59-65, wherein the subject has been determined to be a candidate for ICB therapy.

67. The method of any one of claims 59-66, wherein the subject is currently being treated with ICB therapy, has received at least one ICB therapy, or wherein the subject has not been treated with ICB therapy.

68. The method of any one of claims 59-67, wherein the method further comprises comparing the expression level of CD73 detected to a control.

69. The method of claim 68, wherein the control comprises a biological sample from a subject that does not respond to ICB therapy.

70. The method of claim 68, wherein the control comprises a biological sample from a subject that responds to ICB therapy.

71. The method of claim 69 or 70 wherein the subject is determined to have a higher expression level than the control.

72. The method of claim 69 or 70 wherein the subject is determined to have a lower expression level than the control.

73. The method of claim 69 or 70 wherein the subject is determined to have a level of expression that is not significantly different than the control.

74. The method of any one of claims 66-73, wherein the ICB therapy comprises a monotherapy or combination ICB therapy.

75. The method of any one of claims 66-74, wherein the ICB therapy comprises an inhibitor of PD-1, PDL1, PDL2, CTLA-4, B7-1, and/or B7-2.

76. The method of any one of claims 66-75, wherein the ICB therapy comprises an anti-PD-1 monoclonal antibody and/or an anti-CTLA-4 monoclonal antibody.

77. The method of claim 76, wherein the ICB therapy comprises one or more of nivolumab, pembrolizumab, pidilizumab, ipilimumab or tremelimumab.
